(19) United States
(12) Patent Application Publication
(10) Pub. No.: US 2009/0012026 A1
(43) Pub. Date: Jan. 8, 2009

(54) Association Between the Tdoa Gene and Osteoarthritis

(75) Inventors: Ian David Cook, Cheshire (GB); Angela Flannery, Cheshire (GB); Rose Maciewicz, Cheshire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/067,667

(22) PCT Filed: Sep. 20, 2006
(86) PCT No.: PCT/GB06/03498

§ 371 Date: Mar. 21, 2008

(30) Foreign Application Priority Data
Sep. 23, 2005 (GB) 0519377.6

(51) Int. Cl. A61K 31/7105; C12Q 1/68; G01N 33/53; A61P 19/00; G01N 33/00; C12Q 1/02

(52) U.S. Cl. 514/44; 435/6; 435/7.1; 436/86; 435/7.21; 435/29

(57) ABSTRACT

The present invention arises from the identification of an association between the TDOA gene and osteoarthritis (OA). It therefore relates to diagnostic techniques for determining a patient's susceptibility to develop OA by detecting all or part of the TDOA gene, its precursors or products (mRNA, cDNA, genomic DNA, or protein). In particular, the invention relates to methods and materials for analysing allelic variation in the TDOA gene, and to the use of TDOA polymorphisms in the identification of an individuals' risk to develop OA. The TDOA protein has been found to bind to α-paxillin, a protein involved in integrin signal transduction which itself is a process with strong links to OA. The invention is thus also directed to methods for identifying modulators of OA, which modulate the TDOA gene or interfere with TDOA:α-paxillin binding.

### TECHNICAL FIELD

The inventors have discovered a gene linked to susceptibility to osteoarthritis (OA) using linkage and association analysis. The gene is referred to herein as TDOA. Thus, the present invention identifies a role for TDOA in OA. The present invention therefore relates to diagnostic techniques for the detection of OA, and for determining a patient's susceptibility to develop OA by detecting all or part of this gene, its precursors or products (mRNA, cDNA, genomic DNA, or protein). The invention also relates to polymorphisms within the gene and to methods for their detection. In particular, the invention relates to methods and materials for analysing allelic variation in the TDOA gene, and to the use of TDOA polymorphisms in the identification of an individuals' risk to develop OA.

The polymorphisms of the invention also allow patient stratification. The sub-groups of individuals identified as having increased or decreased risk of developing OA can be used, inter alia, for targeted clinical trial programs and possibly also pharmacogenetic therapies.

The inventors have also discovered that the protein encoded by this gene (TDOA) binds to α-paxillin, a protein involved in integrin signal transduction which itself is a process with strong links to OA. The invention is thus also directed to methods for identifying modulators of OA, which modulators, such as chemical compounds, antisense molecules and antibodies modulate the TDOA gene or interfere with TDOA:α-paxillin binding.

### BACKGROUND TO INVENTION

Osteoarthritic diseases are a result of both mechanical and biologic events that destabilize the normal coupling of degradation and synthesis of articular cartilage chondrocytes and extracellular matrix, and subchondral bone (Creamer and Hochberg, Lancet, 350: 503-508, 1997; Jones and Doherty, Br. Med. J., 310: 457-460, 1995). Although they may be initiated by multiple factors, including genetic, developmental, environmental factors such as metabolic, and traumatic, osteoarthritis diseases involve all of the tissues of the diarthrodial joint. Ultimately, osteoarthritis diseases are manifested by morphologic, biochemical, molecular, and biomechanical changes of both cells and matrix which lead to, amongst others, a softening, fibrillation, ulceration, loss of articular cartilage, sclerosis and eburnation of subchondral bone, osteophytes, and subchondral cysts. Other tissues besides cartilage, such as tendon, ligament, muscle and meniscus can also contribute to disease initiation and progression. When clinically evident, osteoarthritis diseases are characterized by joint pain, tenderness, limitation of movement, crepitus, occasional effusion, and variable degrees of inflammation mainly without overt systemic effects (Kuettner and Goldberg, Osteoarthritic disorders. Rosemont: American Academy of Orthopaedic Surgeons, 1995).

Most epidemiological studies have defined cases of osteoarthritis based on the presence of typical radiographic features. In populations of white North Americans and Northern Europeans, about one-third of adults aged 25-74 years have features of radiographic osteoarthritis involving at least one peripheral joint group: the most common sites are the hands, followed by feet, knees, and hips (Spector and Hochberg, Ann Rheum Dis 53: 43-46, 1994), however, OA in the knees and hips are more clinically important. Age is the strongest determinant of osteoarthritis with prevalence rates for all joints rising with increasing age. Incidence rates also rise with age but there is some evidence that this reaches a plateau in the seventh decade. The mechanism by which age predisposes to osteoarthritis is unclear. Obesity has been strongly linked to osteoarthritis of the knee and, to a lesser extent, the hip, in cross-sectional and prospective studies (Hochberg M C, Lethbridge-Cejku M. Hamerman D, ed. Osteoarthritis: public health implications for an aging population. Baltimore: Johns Hopkins University Press, 1997:169-86).

Current treatment of osteoarthritis is purely to control symptoms because as yet there are no disease-modifying osteoarthritis drugs. The principal measure of treatment efficacy is traditionally pain. The drug of choice is currently paracetamol to which a proportion of patients do not respond but appear to derive benefit from NSAIDs (non-steroidal anti-inflammatory drugs, including COX-2 inhibitors. Topical creams, either a NSAID or capsaicin, can be helpful as either monotherapy or when added to oral analgesics, especially if only a few joints are involved.

Intra-articular steroids are widely used in osteoarthritis, particularly for the knee. There is some evidence to support their efficacy versus pain relief, but only for 1-3 weeks; triamcinolone hexacetonide is the most efficacious preparation. Intra-articular hyaluronic acid has also been shown to be effective in modulating pain in patients with knee osteoarthritis; this therapy requires weekly injections for 3-5 weeks. Genetic components have been demonstrated to exist in osteoarthritis by heritability estimates in twins and by relative risk estimates in siblings. For hand and knee OA, the genetic influence has been calculated at between 39-65% in twins. In a separate study of hand and disc OA the estimates were 56% and 75% respectively. In severe hip OA requiring total hip replacement (THR), the sibling relative risk compared to the population at large has been calculated at 5.6 and this may be an underestimate, since the prevalence of OA in the general population is high. A recent study of female twin pairs showed significant heritability for radiographic features of osteoarthritis at the knee and hip (Spector et al, BMJ. 312: 940-944, 1996). Although mutations in the gene for type II collagen (COL2A1) have been associated with early polyarticular osteoarthritis with mild chondrodysplasia (Knowlton et al New Eng. J. Med. 322: 526-530, 1990), it is unlikely that there is a single gene that fully explains the genetic contribution to osteoarthritis. However, as OA demonstrates considerable clinical heterogeneity there may be specific gene(s) for specific sub classes of OA (Peach et al. Trends Mol. Med. 11(4):186-191, 2005).

There is therefore a desire to identify genes with a significant association to the development of OA. This may enable the development of novel therapies for OA by screening for compounds and other entities, such as antibodies, which modulate the activity of the proteins encoded by the associated genes, or modulate their interaction with other proteins or molecules. Knowledge of the sequence of the associated genes may also enable the development of novel antigene methods to modulate the expression of the associated gene and may also enable the development of novel gene therapy techniques to treat OA. The discovery of associated genes may also assist in developing novel methods for diagnosing OA via (i) analysis of the pattern of genotypes of associated single nucleotide polymorphisms (SNPs), (ii) measuring the levels of the translated mRNA present in affected tissue and (iii) measuring the levels of the protein in affected tissue and biological fluids. It is possible that the diagnosis of OA, or the prediction of predisposition to OA, by these methods may be achieved in patients who do not yet display the classical symptoms of the disease. Such determination of susceptibility to OA or the early detection of disease development may lead to earlier clinical intervention than is currently possible and may lead to more effective treatment of the disease. Such techniques may also allow patient stratification, which may inter alia, be useful in selecting patients for clinical trials.

International Patent publication Number: WO 00/20632 identifies a 20 cM region on chromosome 11 between microsatelite markers D11S4046 and D11S1320 that contains a possible susceptibility locus for OA. This region was identified following a two-stage non-parametric linkage analysis. This revealed three microsatellites on chromosome 11 for which there was evidence of linkage. Finer mapping in and around these microsatellites provided enhanced evidence for linkage and enabled linked regions to be defined. Linkage disequilibrium analysis identified excess transmission of the 258 bp allele of marker D11S937 (allele 11 as listed in the GDB (Genome Database)) in the unstratified data (p<0.001). This data indicated that the OA susceptibility locus on 11q is close to D11S937 and that the 258 bp allele may be present on a predisposing ancestral chromosome. There is however, no disclosure of an association to OA for any single gene nor is any indication as to where such a gene is likely to be located within the 20 cM region disclosed.

The present invention is based on our discovery of an association with OA for a single gene termed TDOA, which maps to a 2.8 cM region within 11q13-14 that incorporates D11S937 at 11q13.5.

TDOA possesses close homology with a human F is clone (accession AK027572) and a mouse clone from Riken (accession AK017384), both of unknown function. Domain searching shows homology in the N-terminus with the tetramerisation domain of a number of potassium channels and also with the BTB/POZ domain found in some zinc-finger proteins and mediates dimerisation.

TDOA is described in WO 01/62923 (Incyte Genomics Inc.) where it is named TRICH-6 and claimed to be a potassium channel, however, the nucleic acid disclosed therein encodes a predicted polypeptide with a 78 amino acid insertion compared to the TDOA sequence identified by the present inventors (SEQ ID NO: 2). The nucleic acid insertion sequence present in the TRICH-6 sequence in WO 01/62923 appears to be an incorrect duplication when compared to the human genome sequence (e.g. EnsEMBL 9.30a1). In addition other patent application describing the TDOA sequence include: human protein encoding cDNA sequence SEQ ID NO:653 HYSEQ INC WO200153455A2; human reproductive system related antigen DNA SEQ ID NO: 9849 Human Genome Sci Inc WO200155320-A2; human secreted protein 5′ EST, SEQ ID NO:3553 Genset EP1033401-A2; and sequence tag and encoded human protein GENSET JP2001269182-A/3546. All patent applications described above are sequence related and no association to OA is described.

The predicted amino acid sequence of TDOA protein is shown in SEQ ID No:2. The genomic sequence of TDOA is disclosed as SEQ ID NO: 1. The cDNA sequence is shown in SEQ ID NO: 3.

### SUMMARY OF THE INVENTION

The present inventors have identified an osteoarthritis disease association with a gene on chromosome 11, and have identified polymorphisms within this gene. The present application therefore provides direct evidence for a role for TDOA in osteoarthritis. The TDOA gene, mRNA and protein sequences derived therefrom may therefore be used as diagnostic or prognostic markers of osteoarthritis, and can be used to design specific probes, or to generate antibodies, capable of detecting the presence of polymorphims of the gene or mRNA, or of measuring the levels of the mRNA or encoded protein present in a test sample, such as a body fluid or cell sample. In addition the gene and protein encoded thereby is a potential target for therapeutic intervention in osteoarthritic disease, for instance in the development of antisense nucleic acid targeted to the mRNA; or more widely in the identification or development of therapeutic agents. The inventors have also discovered that TDOA binds to α-paxillin, a protein involved in integrin signal transduction which itself is a process with strong links to OA (See Peters et al. for a review (Osteoarthritis & Cartilage. 10:831-835 2002)). They have also discovered that over-expression of TDOA in primary human chondrocytes resulted in an abnormal distribution of α-paxillin in the cell, with more of the protein being present in the nucleus, and this leads to changes in gene expression which are consistent with that observed in cells in the affected joints of OA patients. The person skilled in the art is capable of devising screening assays to identify compounds (chemical or biological) that modulate (e.g. activate (agonise) or inhibit (antagonise)) TDOA, its encoded protein or its ability to bind to α-paxillin, which compounds may prove useful as therapeutic agents in treating or preventing osteoarthritis and arthropathies in general.

### DETAILED DESCRIPTION OF THE INVENTION

The following definitions apply to the present application.

“Allele” refers to a particular form of a genetic locus, distinguished from other forms by its particular nucleotide or amino acid sequence.

“Expression” refers to the transcription of a gene's DNA template to produce the corresponding mRNA and translation of this mRNA to produce the corresponding gene product (i.e., a peptide, polypeptide, or protein). The term “activates gene expression” refers to inducing or increasing the transcription of a gene in response to a treatment where such induction or increase is compared to the amount of gene expression in the absence of said treatment. Similarly, the terms “decreases gene expression” or “down-regulates gene expression” refers to inhibiting or blocking the transcription of a gene in response to a treatment and where such decrease or down-regulation is compared to the amount of gene expression in the absence of said treatment.

“Genotype” is an unphased 5′ to 3′ sequence of nucleotide pair(s) found at one or more polymorphic sites in a locus on a pair of homologous chromosomes in an individual.

“Isolated” nucleic acid, as referred to herein, refers to material removed from its original environment (for example, the natural environment in which it occurs in nature), and thus is altered by the hand of man from its natural state. For example, an isolated polynucleotide could be part of a vector or a composition of matter, or could be contained within a cell, and still be “isolated” because that vector, composition of matter, or particular cell is not the original environment of the polynucleotide.

“Locus” refers to a location on a chromosome or DNA molecule corresponding to a gene or a physical or phenotypic feature.

“Polymorphic site” is a position within a locus at which at least two alternative sequences are found in a population.

“Polymorphism” refers to the sequence variation observed in an individual at a polymorphic site. Polymorphisms include nucleotide substitutions, insertions, deletions and microsatellites and may, but need not, result in detectable differences in gene expression or protein function.

Thus according to a first aspect of the invention there is provided a method for identifying a compound of potential therapeutic or prophylactic benefit in treating OA, which method comprises subjecting one or more test compounds to a screen comprising a TDOA polypeptide and determining the ability of the compound or compounds to bind to, block or modulate the polypeptide, or inhibit an activity of the polypeptide. In a particular embodiment, the TDOA polypeptide contains or comprises the amino acid sequence shown in SEQ ID NO: 2, or a homologue thereof or a fragment of either.

According to a further aspect of the invention there is provided a method for identifying a compound of potential therapeutic or prophylactic benefit, which method comprises measuring the ability of a test compound to interfere with or inhibit the binding of TDOA to α-paxillin. A compound that is capable of interfering or inhibiting the binding of TDOA to α-paxillin is one that may have potential therapeutic or prophylactic benefit. In a particular embodiment the compound may have potential therapeutic or prophylactic benefit in treating or preventing OA.

The term “fragment” as used herein refers to a subsequence of the full length sequence that comprises at least 25, preferably at least 50, more preferably at least 100 consecutive amino acids of the sequence depicted in SEQ ID NO: 2, preferably the fragment is a polypeptide that is the TDOA protein with either or both C-terminal and N-terminal truncations.

It is understood that the polypeptide for use in the invention may be both a fragment and a homologue of the TDOA protein.

In a preferred embodiment, the screening methods of the invention are carried out using a polypeptide comprising an amino acid sequence as depicted in SEQ ID NO: 2, or a sequence possessing, in increasing order of preference, at least 80%, 85%, 90%, 95%, 97%, 98% and 99% amino acid sequence identity thereto. Such variants are herein referred to as “homologues”. Suitable homologues for use in the invention include those whose nucleic acids hybridise to a nucleic acid sequence encoding the polypeptide depicted in SEQ ID NO: 2 under stringent hybridisation conditions, which homologues retain a function of the TDOA protein.

The sequence identity between two sequences can be determined by pair-wise computer alignment analysis, using programs such as, BestFit, Gap or FrameAlign. The preferred alignment tool is BestFit. In practise, when searching for similar/identical sequences to the query search, from within a sequence database, it is generally necessary to perform an initial identification of similar sequences using suitable software such as Blast, Blast2, NCBI Blast2, WashU Blast2, FastA, Fasta3 and PILEUP, and a scoring matrix such as Blosum 62. Such software packages endeavour to closely approximate the “gold-standard” alignment algorithm of Smith-Waterman. Thus, the preferred software/search engine programme for use in assessing similarity, i.e how two primary polypeptide sequences line up is Smith-Waterman. Identity refers to direct matches, similarity allows for conservative substitutions.

Allelic variants or versions of the TDOA protein may exist within the human population, particularly between distinct ethnic groups. A further aspect of the invention involves the selection and use of the appropriate version of the TDOA protein to be included in screens so as to discover compounds capable of altering the activity of said TDOA version in vivo, or alter its ability to bind to itself (i.e. form multimers) or to α-paxillin. Investigators may wish to screen their compounds against the most prevalent version of the TDOA protein and also against the less frequent versions of the TDOA protein in order to detect any differential pharmacological activity between the various versions of the target. A further aspect of the invention is the screening of various ethnic based populations to measure the allele frequencies of the single nucleotide polymorphisms (mutations) in the TDOA gene within said populations. This information may be of value in estimating the efficacy of new compounds capable of altering the activity of TDOA within these populations and in particular in estimating the proportion of the population that may not respond to the therapy.

Candidate compounds may be identified from a variety of sources, for example, cells, cell-free preparations, chemical libraries, peptide and gene libraries, and natural product mixtures. Chemical libraries include combinatorial chemistry libraries and, in particular, a combinatorial chemical library comprising compounds that interact with GPCRs. Such antagonists or inhibitors so-identified may be natural or modified substrates, ligands, receptors, enzymes, antibodies (as described above) etc., as the case may be, of the TDOA protein; or may be structural or functional mimetics thereof (see Coligan et al., Current Protocols in Immunology 1(2):Chapter 5 (1991)).

Techniques such as analytical centrifugation affinity binding studies involving chromatography or electrophoresis can be used to detect molecules which interact directly with TDOA. Other techniques that allow the identification of protein-protein interactions include immunoprecipitation and yeast two hybrid studies.

Compounds having inhibitory, activating, or modulating activity can be identified using in vitro and in vivo assays for TDOA activity (e.g. binding to α-paxillin) and/or expression, e.g., ligands, agonists, antagonists, and their homologs and mimetics.

Further aspects of the invention arise from the identification of polymorphisms (mostly single base change polymorphisms/mutations) in TDOA as outlined in Table 2.

Polymorphism refers to the occurrence of two or more genetically determined alternative alleles or sequences within a population. A polymorphic marker is the site at which divergence occurs. Preferably markers have at least two alleles, each occurring at frequency of greater than 1%, and more preferably at least 10%, 15%, 20%, 30% or more of a selected population.

Single nucleotide polymorphisms (SNP) are generally, as the name implies, single nucleotide or point variations that exist in the nucleic acid sequence of some members of a species. Such polymorphic variations within the species are generally regarded to be the result of spontaneous mutation throughout evolution. The mutated and normal sequences co-exist within the species' population sometimes in a stable or quasi-stable equilibrium. At other times the mutation may confer some advantage to the species and with time may be incorporated into the genomes of all or a majority of members of the species.

Some SNPs alter protein coding sequences, in which case, one of the polymorphic protein forms may possess a different amino acid which may give rise to the expression of a variant protein and, potentially, a genetic disease. These changes in function may be mediated by several mechanisms including, but not limited to, alterations in protein folding, alterations in ligand and substrate binding affinity and alterations in membrane binding affinity and may lead to gain of activity/function or loss of activity/function for the protein in vivo. Such alterations in the activity/function of the protein in vivo may be of clinical significance in the development of OA. Alteration to the amino acid sequence of the protein may also affect the efficacy of drug therapy for OA by altering the specificity between protein and compounds selected by screening to modulate its activity. Other SNPs occur in regulatory regions of the gene, in promoters, splice donor or acceptor sites, in 3′UTRs or 5′UTRs. Variants in such sites may alter the function and/or the expression of the gene in a similar way to, but not limited to, protein coding variants. Thus compounds selected by screening may have different efficacies in modulating the activity of protein in different individuals according to the versions of the gene that they carry. In particular an individual who is homozygous for a less common variant of the gene may not respond well to a therapy developed by screening compounds against the dominant variant.

The use of knowledge of polymorphisms to help identify patients most suited to therapy with particular pharmaceutical agents is often termed “pharmacogenetics”. Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. Clinical Chemistry, 43:254, 1997; Marshall Nature Biotechnology. 15:1249, 1997; International Patent Application WO 97/40462, Spectra Biomedical; and Schafer et al, Nature Biotechnology. 16:33, 1998.

A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms that are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine (De Stefano et al., Ann Hum Genet 62:481-90, 1998; and, Keightley et al., Blood 93:4277-83, 1999).

Clinical trials have shown that patient response to treatment with pharmaceuticals is often heterogeneous. Thus there is a need for improved approaches to pharmaceutical agent design and therapy, as well as clinical trial design (including patient selection). The ability to stratify patients into groups based on their likely response to the drug (e.g. in terms of safety or efficacy) would be extremely useful in the quest to identify appropriate drugs of therapeutic value.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The presence of a particular nucleic acid base at a polymorphism position will be represented by the base following the polymorphism position. For (a hypothetical) example, the presence of adenine at position 300 will be represented as: 300A.

We provide examples of polymorphisms (mutations) that potentially affect the sequence of the TDOA protein. These polymorphisms are shown in Tables 2 and 3.

Single nucleotide polymorphisms (mutations) in the promoter and UTR regions may also affect the transcription and expression of the TDOA gene leading to either increased or decreased levels of expression or to unregulated activity of the TDOA protein in vivo. Such alterations in the level of expression of the TDOA protein in vivo may result in a gain or loss of function that is of clinical significance. Recently, it has been reported that even polymorphisms that do not result in an amino acid change can cause different structural folds of mRNA with potentially different biological functions (Shen et al., Proc Natl Acad Sci USA 96:7871-7876, 1999).

In one embodiment of the invention the screening methods described herein utilise a TDOA protein variant which is transcribed from the nucleic acid sequence shown in SEQ ID NO:1 or SEQ ID NO:3, and which incorporates one or more of the polymorphisms identified herein and listed in Tables 2 and 3.

The polymorphisms (mutations) may also be used as diagnostic markers of predisposition to disease. Genotyping polymorphisms (particularly SNPs) in populations suffering from OA and in control populations not suffering from OA but matched for factors including, but not limited to, racial ancestry, country of origin, sex, age and body mass index may allow investigators to identify increased risk factors associated with the development of OA disease according to the inheritance of certain polymorphisms genotypes or haplotypes which are more prevalent in populations with OA compared to their incidence in the corresponding control populations. This may enable screening for individuals at increased risk of developing OA by measuring the genotypes and haplotypes of these polymorphisms within non-symptomatic individuals. We have discovered several polymorphisms in the TDOA gene that may be useful for the diagnosis of increased risk to develop OA. These polymorphisms are shown in Table 2. Preferred polymorphisms are those shown in Table 3. {***+LD table for GT filling only}

The screening methods according to the invention may be operated using conventional procedures, for example by bringing the test compound or compounds to be screened and an appropriate substrate into contact with the polypeptide, or a cell capable of producing it, or a cell membrane preparation thereof, and determining affinity for the polypeptide in accordance with standard techniques.

Any compound identified in this way may prove useful in the treatment of OA in humans and/or other animals. The invention thus extends to a compound selected through its ability to regulate the activity of the TDOA protein in vivo as primarily determined in a screening assay utilising the polypeptide containing an amino acid sequence shown in SEQ ID NO: 2, or a homologue or fragment thereof, or a gene coding therefore (such as that disclosed in SEQ ID NO: 1) for use in the treatment of a disease in which the over- or under-activity/function or unregulated activity/function of the protein is implicated.

According to a further aspect of the invention there is provided a screening assay or method for identifying potential disease modifying anti-OA drugs (DMOAD) comprising contacting an assay system capable of detecting the effect of a test compound against expression level of TDOA, with a test compound and assessing the change in expression level of TDOA. In a particular embodiment a potential therapeutic compound for treating an OA disorder is one capable of reducing the expression level of TDOA.

Compounds that modulate the expression of DNA or RNA of TDOA polypeptides may be detected by a variety of assay systems. A suitable assay system may be a simple “yes/no” assay to determine whether there is a change in expression of a reporter gene, such as beta-galactosidase, luciferase, green fluorescent protein or others known to the person skilled in the art (reviewed by Naylor, Biochem. Pharmacol. 58: 749-57, 1999). The assay system may be made quantitative by comparing the expression or function of a test sample with the levels of expression or function in a standard sample. Systems in which transcription factors are used to stimulate a positive output, such as transcription of a reporter gene, are generally referred to as “one-hybrid systems” (Wang and Reed. Nature. 364:121-126, 1993). Using a transcription factor to stimulate a negative output (growth inhibition) may thus be referred to as a “reverse one-hybrid system” (Vidal et al, 1996, supra). Therefore, in an embodiment of the present invention, a reporter gene is placed under the control of the TDOA promoter.

In a further aspect of the invention we provide a cell or cell line comprising a reporter gene under the control of the TDOA promoter.

According to another aspect of the present invention there is provided a method of screening for a compound potentially useful for treatment of OA, which comprises assaying the compound for its ability to modulate the activity or amount of TDOA. Preferably the assay is selected from:
i) measurement of TDOA activity using a cell, cell line or tissue which expresses the TDOA polypeptide or using purified TDOA polypeptide; and
ii) measurement of TDOA transcription or translation in the cell, cell line or tissue extract expressing the TDOA polypeptide

In a particular embodiment a potential therapeutic compound for treating an OA disorder is one capable of reducing the activity or amount of TDOA.

The “TDOA polypeptide” refers to the TDOA protein, a homologue thereof, or a fragment of either.

Thus, in a further aspect of the invention, cell cultures expressing the TDOA polypeptide can be used in a screen for therapeutic agents. Effects of test compounds may be assayed by changes in mRNA or protein of TDOA. As described below, cells (i.e. mammalian, bacterial etc) can be engineered to express the TDOA polypeptide.

Thus, according to a further aspect of the invention there is provided a method of testing potential therapeutic agents for the ability to suppress OA disorder phenotype comprising contacting a test compound with a cell engineered to express the TDOA polypeptide; and determining whether said test compound suppressed expression of the TDOA polypeptide.

We also provide a method for identifying inhibitors of transcription of TDOA, which method comprises contacting a potential therapeutic agent with a cell or cell line as described above and determining inhibition of TDOA transcription by the potential therapeutic agent by reference to a lack of or reduction in expression of the reporter gene.

According to a further aspect of the invention there is provided a method for identifying a compound of potential therapeutic or prophylactic benefit, which method comprises measuring the ability of a test compound to interfere with or inhibit the binding of TDOA to α-paxillin or to itself. A compound that is capable of interfering or inhibiting the binding of TDOA to α-paxillin or to itself is one that may have potential therapeutic or prophylactic benefit. In a particular embodiment this will be in treating or preventing OA.

There are many different techniques for measuring protein:protein interactions through which potential therapeutics could be identified that interfere with the interaction between TDOA and α-paxillin. Examples of such techniques include yeast 2-hybrid (Fields and Song, Nature. 340(6230):245-6, 1989), mammalian 2-hybrid (Dang, C. V., et al. Mol. Cell Biol. 11: 954-962, 1991), surface plasma resonance (see for example L. T. Vassilev et al., Science. 303:844-8, 2004), fluorescence resonance energy transfer (FRET; for a review see Matyus L., J Photochem Photobiol B. 12(4):323-37, 1992) or co-immunoprecipitation (see for example Adams and Ohh. Nature Methods. 2:475-476, 2005). Such techniques could be used to identify substances that disrupt the interaction between full-length TDOA and either full-length α-paxillin, or truncations and deletions of α-paxillin which incorporate the region originally identified by yeast 2-hybrid screening. The α-paxillin gene sequence is disclosed in EMBL database under accession number: HS14588. The encoded protein sequence is disclosed as SEQ ID NO: 8. The amino acid sequence of an active fragment of α-paxillin, identified herein, is shown in SEQ ID NO: 9. Additionally, these approaches could also be used to identify substances that disrupt the interaction of full-length TDOA with itself.

Any convenient test compound or library of test compounds may be used in conjunction with a test assay. Particular test compounds include low molecular weight chemical compounds (preferably with a molecular weight less than 1500 daltons) suitable as pharmaceutical or veterinary agents for human or animal use, or compounds for non-administered use such as cleaning/sterilising agents or for agricultural use. Test compounds may also be biological in nature, such as antibodies.

According to a further aspect of the invention there is provided a compound identified by a screening method as defined herein.

According to another aspect of the present invention there is provided use of a compound able to modulate the activity or amount of TDOA in the preparation of a medicament for the treatment of OA. Modulation of the amount of TDOA by a compound may be brought about for example through altered gene expression level or message stability. Modulation of the activity of TDOA by a compound may also be brought about for example through compound binding to the TDOA protein. In one embodiment, modulation of TDOA comprises use of a compound able to reduce the activity or amount of TDOA. In another embodiment, modulation of TDOA comprises use of a compound able to increase the activity or amount of TDOA.

The inventors have discovered that TDOA can bind to itself as multimers and that TDOA most likely binds to α-paxillin as a multimer. This opens up the opportunity to screen for compounds with the ability to disrupt TDOA multimerisation and resulting loss of binding to α-paxillin.

According to another aspect of the present invention there is provided use of a compound able to inhibit binding of TDOA to α-paxillin or inhibit TDOA multimerisation in the preparation of a medicament for the treatment of OA. In this context the term inhibit includes completely inhibiting or preventing binding as well as reducing the amount of binding.

It will be appreciated that the term ‘for the treatment of OA, and variations thereon, includes therapeutic and prophylactic (preventative) treatment.

According to another aspect of the present invention there is provided a method of preparing a pharmaceutical composition which comprises:

i) identifying a compound as useful for treatment of OA according to a screening method as described herein; and
ii) mixing the compound or a pharmaceutically acceptable salt thereof with a pharmaceutically acceptable excipient or diluent.

According to a further aspect of the invention there is provided a method of treatment of a patient suffering from OA comprising administration to said patient of an effective amount of a compound identified according to a screening method of the invention or a composition prepared by the method described herein.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intra-articular, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient, which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound will naturally vary according to the nature and severity of the conditions, the age and sex of the animal or patient and the route of administration, according to well known principles of medicine.

In using a compound for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration and intra-articular injection are however preferred.

Having identified that the TDOA gene is implicated in OA, this presents many molecular diagnostic opportunities. It is known to persons skilled in the art that clinically significant information may be obtained by the measurement of the levels of nucleic acids, proteins or other analytes that occur within biological samples. When nucleic acids, proteins or other analytes occur in polymorphic form then there may also be diagnostic utility in by identifying which of the various versions of said polymorphic nucleic acids, proteins or other analytes occur within a sample.

An investigator may wish to measure the levels of TDOA protein or to measure the levels of TDOA mRNA transcript present in a sample. An investigator may also wish to perform nucleic acid sequence analyses to detect variant nucleotides (e.g. SNPs) present within the sample, these analyses may be performed on either the DNA or RNA fraction of the sample and are well known to the person skilled in the art. An investigator may also wish to perform protein sequence analysis either directly by degradation based techniques which are well known in the art or indirectly by molecular recognition techniques including immunoassay or by techniques based on detecting changes in the physical characteristics of the protein such as functional or substrate specificity assays or iso-electric focusing.

According to a further aspect of the invention there is provided a method for diagnosing or prognosing or monitoring OA, comprising testing a biological sample for aberrant levels of TDOA.

The term “aberrant levels” refers to levels that are outside the normal range. The normal range can be determined by testing many normal tissues or may be determined from a side by side comparison of the test sample with the normal or control sample. For the purposes of this application, aberrant expression refers to a statistically significant difference in level of nucleic acid in a disease sample compared to control normal. Nucleic acid as used herein refers to both RNA and DNA. “Statistically significant” generally means significant at the 90% confidence level, preferably 95% confidence level, more preferably 98% confidence level and especially 99% confidence level.

The test sample is conveniently a sample of synovial fluid, blood (e.g. serum, plasma etc.), buccal scrape, urine or other body fluid or tissue obtained from an individual.

The invention lies in the identification of the gene identified herein being linked to OA disease prevalence. Accordingly, in part, the invention is directed to any diagnostic method capable of assessing the differential expression level, relative to expression in control tissues, of the TDOA gene identified herein, either alone or as a panel. In particular, such methods include assessment of mRNA transcript levels and/or protein levels where the presence of aberrant expression levels of the gene indicate the presence of OA or an increased likelihood to develop the disorder.

As noted above, in one embodiment the diagnostic/detection methods of the invention are employed to detect the presence of one or more polymorphisms of TDOA.

According to another aspect of the present invention there is provided a method for the diagnosis of a polymorphism in TDOA, which method comprises determining the sequence of the human at least one polymorphic position and determining the status of the human by reference to the polymorphism in TDOA. Preferred polymorphic positions are one or more of: 30983, 38160, 12523 . . . 12542 and 32945 (each according to SEQ ID NO: 1), or a polymorphism in linkage disequilibrium above D′ 0.9 therewith.

The term “diagnosis of a polymorphism” refers to determination of the genetic status of an individual at a polymorphic position (in which the individual may be homozygous or heterozygous at each position).

According to another aspect of the present invention there is provided a method for the diagnosis of, or susceptibility to develop, osteoarthritis in a human, which method comprises determining the sequence of the nucleic acid of the human at one or more of positions: 30983, 38160, 12523 . . . 12542 and 32945 (each according to SEQ ID NO: 1), or a polymorphism in linkage disequilibrium above D′ 0.9 therewith, and determining the status of the human by reference to polymorphism in the TDOA gene. In a particular embodiment, this method is used to assess the predisposition and/or susceptibility to develop OA in the human (e.g. assessing the risk of developing OA at a future date). The particular alleles that show an association with OA are identified in Table 3.

In particular embodiments, the presence of an adenine at position 30983 and/or a guanine at position 38160 and or a deletion of the sequence from positions 12523-12542 (each according to the location in SEQ ID NO: 1) is indicative of an increased risk of developing OA (e.g. predisposition to OA). It should further be noted that detection of the nucleotide in the complement strand to SEQ ID NO:1 that base-pairs with the nucleotide at a particular position (e.g. position 30983 of SEQ ID NO:1) is of course within the scope of the claimed invention.

According to another aspect of the present invention there is provided a method for genotyping an individual, which method comprises determining the sequence of the nucleic acid of the human at one or more of positions: 30983, 38160, 12523 . . . 12542 and 32945 (each according to SEQ ID NO: 1), or a polymorphism in linkage disequilibrium above D′ 0.9 therewith, and determining the genotype status of the human by reference to polymorphism in the TDOA gene. The genotype status of the individual can then be used to gauge the individual's risk of developing OA.

In particular embodiments, these particular aspects are applied to the detection or one or more of the polymorphisms identified in Table 2.

The term “status” refers to the genetic status of the human as detected by potential sequence variation at defined positions of a polynucleotide or corresponding protein.

Determining the status of the individual can simply be determining the genotype of the individual (i.e. the identity of the allele at the particular polymorphism) and/or translating that information into a clinical significance, (e.g. assigning a likely risk of developing OA).

The polymorphisms of the invention demonstrate significant association to OA. However, the person skilled in the art will appreciate that OA is a polygenic disease and therefore, a diagnostic test consisting solely of a polymorphism (e.g. a SNP), or even a haplotype comprising one or more polymorphisms of the invention will not be diagnostic of disease occurrence for any particular individual. Nevertheless, in line with future developments we envisage that the polymorphisms of the present invention could form part of a panel of markers that in combination will be predictive of disease or disease susceptibility for an individual, within normal clinical standards sufficient to influence clinical practice.

According to another aspect of the invention there is provided a method of determining if an individual is predisposed to OA, the method comprising determining a presence or absence, in a homozygous or heterozygous form, of at least one OA-associated genotype in the TDOA locus or in neighbouring loci of the individual, said neighbouring loci being in linkage disequilibrium with said TDOA locus, thereby determining if the individual is predisposed to OA.

According to another aspect of the invention there is provided a method for assessing the predisposition and/or susceptibility of an individual to OA, which method comprises:
(i) providing a nucleic acid sample that has been removed from the individual;
(ii) determining the identity of one or more nucleotides at a polymorphic site in the TDOA gene of the individual; and
(iii) determining the status of the individual by reference to said one or more nucleotides.

In another aspect of the invention there is provided a method for the diagnosis of OA or determining susceptibility to develop OA, which method comprises:
i) obtaining a protein or nucleic acid containing sample that has been removed from an individual; detecting the presence or absence of a variant TDOA on the basis of the presence of a polymorphic amino acid within the TDOA protein, or a polymorphic base within the TDOA gene sequence; and,
ii) determining the status of the human by reference to the presence or absence of a polymorphism in TDOA.

It will be apparent to the person skilled in the art that there are a large number of analytical procedures that may be used to detect the presence or absence of variant nucleotides at one or more polymorphic positions of the invention. In general, the detection of allelic variation requires a mutation discrimination technique, optionally an amplification reaction and optionally a signal generation system. List 1 lists a number of mutation detection techniques, some based on the polymerase chain reaction (PCR). These may be used in combination with a number of signal generation systems, a selection of which is listed in List 2. Further amplification techniques are listed in List 3. Many current methods for the detection of allelic variation are reviewed in Nollau et al., Clin. Chem. 43:1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

TABLE 1
Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| CMC | Chemical mismatch cleavage |
| Bp | base pair |
| COPS | Competitive oligonucleotide priming system |
| DGGE | Denaturing gradient gel electrophoresis |
| FRET | Fluorescence resonance energy transfer |
| LCR | Ligase chain reaction |
| INDEL | Polymorphic Insertion or deletion of a specified nucleic acid |
| | sequence |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SERRS | Surface enhanced raman resonance spectroscopy |
| SNP | Single nucleotide polymorphism |
| SSCP | Single-strand conformation polymorphism analysis |
| SSR | Self sustained replication |
| TGGE | Temperature gradient gel electrophoresis |
| 3′ UTR | 3′ untranslated region |

### List 1—Mutation Detection Techniques

General: DNA sequencing, Sequencing by hybridisation
Scanning: PTT*, SSCP, DGGE, TGGE, Cleavase, Heteroduplex analysis, CMC, Enzymatic mismatch cleavage
* Note: not useful for detection of promoter polymorphisms.
Hybridisation Based Solid phase hybridisation: Dot blots, MASDA, Reverse dot blots, Oligonucleotide arrays (DNA Chips)
Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)
Extension Based: ARMS™-allele specific amplification (as described in European patent No. EP-B-332435 and U.S. Pat. No. 5,595,890), ALEX™—European Patent No. EP 332435 B1 (Zeneca Limited), COPS—Gibbs et al (1989), Nucleic Acids Research, 17, 2347.

### Incorporation Based Mini-sequencing, APEX

Restriction Enzyme Based: RFLP, Restriction site generating PCR

### Ligation Based: OLA

Other: Invader assay, Hybridisation protection assay

### List 2—Signal Generation or Detection Systems

Fluorescence: FRET, Fluorescence quenching, Fluorescence polarisation —United Kingdom Patent No. 2228998 (Zeneca Limited)
Other: Chemiluminescence, Electrochemiluminescence, Raman, Radioactivity, Colorimetric, Mass spectrometry, SERRS—WO 97/05280 (University of Strathclyde).

### List 3—Further Amplification Methods

SSR, NASBA, LCR, SDA, b-DNA

Preferred mutation detection techniques include ARMS™-allele specific amplification, Taqman™, Mini sequencing, sequencing, RFLP, ALEX™, OLA, restriction site based PCR and FRET techniques.

Particularly preferred methods include ARMS™-allele specific amplification, OLA and RFLP based methods. ARMS™-allele specific amplification is an especially preferred method.

ARMS™-allele specific amplification (described in European patent No. EP-B-332435, U.S. Pat. No. 5,595,890 and Newton et al. (Nucleic Acids Research, 17:2503, 1989)), relies on the complementarity of the 3′ terminal nucleotide of the primer and its template. The 3′ terminal nucleotide of the primer being either complementary or non-complementary to the specific mutation, allele or polymorphism to be detected. There is a selective advantage for primer extension from the primer whose 3′ terminal nucleotide complements the base mutation, allele or polymorphism. Those primers which have a 3′ terminal mismatch with the template sequence severely inhibit or prevent enzymatic primer extension. Polymerase chain reaction or unidirectional primer extension reactions therefore result in product amplification when the 3′ terminal nucleotide of the primer complements that of the template, but not, or at least not efficiently, when the 3′ terminal nucleotide does not complement that of the template.

It will be appreciated that the test sample may equally be a nucleic acid sequence corresponding to the sequence in the test sample, that is to say that all or a part of the region in the sample nucleic acid may firstly be amplified using any convenient technique e.g. polymerase chain reaction (PCR), before analysis. The nucleic acid may be genomic DNA or fractionated or whole cell RNA. In one embodiment the RNA is whole cell RNA and is used directly as the template for labelling a first strand cDNA using random primers or poly A primers. The nucleic acid or protein in the test sample may be extracted from the sample according to standard methodologies (Sambrook et al. “Molecular Cloning—A Laboratory manual”, second edition. Cold Spring Harbor, N.Y. (1989)).

It will be apparent that the gene sequence disclosed for TDOA (as depicted in SEQ ID NO: 1) is a representative sequence. In normal individuals there are two copies of each gene, a maternal and paternal copy, which will likely have some sequence differences, moreover within a population there will exist numerous allelic variants of the gene sequence (natural allelic variants). It will be appreciated that the diagnostic methods and other aspects of this invention extend to the detection etc. of any of these sequence variants. Preferred sequence variants are those that possess at least 90% and preferably at least 95% sequence identity (nucleic acid or amino acid) to TDOA depicted in SEQ ID No. 1 or 2. Nucleic acid sequence identity can also be gauged by hybridisation studies whereby, under stringent hybridisation and wash conditions, only closely related sequences (for example, those with >90% identity) are capable of forming a hybridisation complex.

The amino acid sequence method for diagnosis is preferably one which is determined by immunological methods such as enzyme linked immunosorbent assay (ELISA).

The levels of the TDOA can be assessed from relative amounts of mRNA, cDNA, genomic DNA or polypeptide sequence present in the test sample. Where RNA is used, it may be desired to convert the RNA to a complementary cDNA and during this process it may be desirable to incorporate a suitable detectable label into the cDNA.

In a particular embodiment the method of the invention relies on detection of mRNA transcript levels. This involves assessment of the relative mRNA transcript levels of TDOA in a sample, and comparison of sample data to control data. The gene transcript can be detected individually, or, is preferably detected amongst a panel of other disease-linked gene TDOA from which a transcript profile can be generated. Levels of TDOA mRNA in the test sample can be detected by any technique known in the art. These include Northern blot analysis, reverse transcriptase-PCR amplification (RT-PCR), microarray analysis and RNAse protection.

In one embodiment, levels of TDOA RNA in a sample can be measured in a Northern blot assay. Here, tissue RNA is fractionated by electrophoresis, fixed to a solid membrane support, such as nitrocellulose or nylon, and hybridised to a probe or probes capable of selectively hybridising with the TDOA RNA to be detected. The actual levels may be quantitated by reference to one or more control housekeeping genes. Probes may be used singly or in combination. This may also provide information on the size of mRNA detected by the probe. Housekeeping genes are genes that are involved in the general metabolism or maintenance of the cell, and are considered to be expressed at a constant level irrespective of cell type, physiological state or stage in the cell cycle. Examples of suitable housekeeping genes are: beta actin, GAPDH, histone H3.3 or ribosomal protein L13 (Koehler et al., Quantitation of mRNA by Polymerase Chain Reaction. Springer-Verlag, Germany (1995)).

To gauge relative expression levels, a control sample can be run alongside the test sample or, the test result/value can be compared to TDOA expression levels expected in a normal or control tissue. These control values can be generated from prior test experiments using normal or control tissues, to generate mean or normal range values for TDOA.

In another embodiment, the TDOA nucleic acid in a tissue sample is amplified and quantitatively assayed. The polymerase chain reaction (PCR) procedure can be used to amplify specific nucleic acid sequences through a series of iterative steps including denaturation, annealing of oligonucleotide primers (designed according to the sequence disclosed in SEQ ID NO. 1), and extension of the primers with DNA polymerase (see, for example, Mullis, et al., U.S. Pat. No. 4,683,202; Loh et al., Science 243:217 (1988)). In reverse transcriptase-PCR (RT-PCR) this procedure is preceded by a reverse transcription step to allow a large amplification of the number of copies of mRNA (Koehler et al., supra). Other known nucleic acid amplification procedures include transcription-based amplification systems (TAS) such as nucleic acid based sequence application (NASBA) and 3SR (Kwoh et al., Proc Natl. Acad Sci USA 86:1173 (1989), Gingeras et al., PCT application WO 88/10315), the ligase chain reaction (LCR, see European Application No. 320308), Strand Displacement Amplification (SDA), “race”, “one sided PCR” and others (Frohman, PCR Protocols: a Guide to Methods and Applications. Academic Press, NY (1990); Ohara et al., Proc Natl. Acad Sci USA 86:5673-5677, 1989). Quantitation of RT-PCR products can be done while the reaction products are building up exponentially, and can generate diagnostically useful clinical data. In one embodiment, analysis is carried out by reference to one or more housekeeping genes which are also amplified by RT-PCR. Quantitation of RT-PCR product may be undertaken, for example, by gel electrophoresis visual inspection or image analysis, HPLC (Koehler et al., supra) or by use of fluorescent detection methods such as intercalation labelling, Taqman probe (Higuchi et al., Biotechnology 10:413-417, 1992), Molecular Beacon (Piatek et al., Nature Biotechnol. 4:359-363, 1998), primer or Scorpion primer (Whitcombe et al., Nature Biotech 17:804-807, 1999); or other fluorescence detection method, relative to a control housekeeping gene or genes as discussed above.

TDOA RNA measurements can also be carried out on synovial fluid, blood or serum samples. Preferably, the RNA is obtained from a peripheral blood sample. In the case of soluble RNA in the blood serum, the low abundance of mRNA expected would necessitate a sensitive test such as RT-PCR (Kopreski et al., Clin Cancer Res 5:1961-5, 1999). A whole blood gradient may be performed to isolate nucleated cells and total RNA is extracted such as by the Rnazole B method (Tel-Test Inc., Friendsworth, Tex.) or by modification of methods known in the art such as described in Sambrook et al., (supra).

In a particular embodiment of the invention, the diagnosis/detection method of the invention involves assessing TDOA transcript levels using microarray analysis. Microarray technology makes it possible to simultaneously study the expression of many thousands of genes in a single experiment. Analysis of gene expression in human tissue (e.g. biopsy or post mortem tissue) can assist in the diagnosis and prognosis of disease and the evaluation of risk for disease. A comparison of levels of expression of various genes from patients with defined pathological disease conditions with normal patients enables an expression profile, characteristic of disease, to be created.

Probes are made that selectively hybridise to the sequences of the target TDOA gene in the test sample. These probes, perhaps together with other probes and control probes, are bound at discrete locations on a suitable support medium such as a nylon filter or microscope slide to form a transcript profiling array. The diagnostic method involves assessing the relative mRNA transcript level of the TDOA in a clinical sample. This can be done by radioactively labelling, or non-radioactively labelling the tissue mRNA, which can be optionally purified from total RNA, in any of a number of ways well known to the art (Sambrook et al., supra). The probes can be directed to any part, or all of, the target TDOA mRNA.

In another embodiment of the invention, total TDOA RNA or DNA is quantified and compared to levels in control tissue or expected levels from pre tested standards. DNA and/or RNA may be quantified using techniques well known in the art. Messenger RNA is often quantitated by reference to internal control mRNA levels within the sample, often relative to housekeeping genes (Koehler et al., supra).

In a preferred embodiment hybridisation signals generated are measured by computer software analysis of images on phosphorimage screens exposed to radioactively labelled tissue RNA hybridised to a microarray of probes on a solid support such as a nylon membrane. In another, quantities are measured by densitometry measurements of radiation-sensitive film (e.g. X-ray film), or estimated by visual means. In another embodiment quantities are measured by use of fluorescently labelled probe, which may be a mixture of biopsy and normal RNA differentially labelled with different fluorophores, allowing quantities of TDOA mRNA to be expressed as a ratio versus the normal level. The solid support in this type of experiment is generally a glass microscope slide, and detection is by fluorescence microscopy and computer imaging.

The detection of specific interactions may be performed by detecting the positions where the labelled target sequences are attached to the array. Radiolabelled probes can be detected using conventional autoradiography techniques. Use of scanning autoradiography with a digitised scanner and suitable software for analysing the results is preferred. Where the label is a fluorescent label, the apparatus described, e.g. in International Publication No. WO 90/15070; U.S. Pat. No. 5,143,854 or U.S. Pat. No. 5,744,305 may be advantageously applied. Indeed, most array formats use fluorescent readouts to detect labelled capture:target duplex formation. Laser confocal fluorescence microscopy is another technique routinely in use (Kozal et al., Nature Medicine 2:753-759, 1996). Mass spectrometry may also be used to detect oligonucleotides bound to a DNA array (Little et al, Analytical Chemistry 69: 4540-4546, 1997). Whatever the reporter system used, sophisticated gadgetry and software may be required in order to interpret large numbers of readouts into meaningful data (such as described, for example, in U.S. Pat. No. 5,800,992 or International Publication No. WO 90/04652).

In a preferred embodiment of the microarray test, the TDOA RNA measurement is generated as a value relative to an internal standard (i.e. a housekeeping gene) known to be constant or relatively constant. The histone H3.3 and ribosomal protein L19 housekeeping genes have been shown to be cell-cycle independent and constitutively expressed in all tissues (Koehler et al., supra). For normalisation of data, several different housekeeping genes can be used to generate an average housekeeping measurement.

A microarray or RT-PCR test to detect OA disorder or susceptibility thereto can be used where tissue samples containing mRNA are available.

Samples for RNA extraction must be treated promptly to avoid RNA degradation (Sambrook et al., supra). This entails either prompt extraction using e.g. phenol-based reagents or snap freezing in e.g. liquid Nitrogen. Samples can be stored at −70° C. or less until RNA can be extracted at a later date. Proprietary reagents are available which allow tissue or cells to be conveniently stored for several days at room temperature and up to several months at 4° C. (e.g. RNAlater, Ambion Inc., TX). Prior to extraction, methods such as grinding, blending or homogenisation are used to dissipate the tissue in a suitable extraction buffer. Typical protocols then use solvent extraction and selective precipitation techniques.

In another embodiment oligonucleotide probe(s) capable of selectively hybridising to TDOA nucleic acid, can be used to detect levels of TDOA gene expression.

Convenient DNA sequences for use in the various aspects of the invention may be obtained using conventional molecular biology procedures, for example by probing a human genomic or cDNA library with one or more labelled oligonucleotide probes containing 10 or more contiguous nucleotides designed using the nucleotide sequences described here. Alternatively, pairs of oligonucleotides one of which is homologous to the sense strand and one to the antisense strand, designed using the nucleotide sequences described here to flank a specific region of DNA may be used to amplify that DNA from a cDNA library.

Primers or probes for use in any of the methods of the invention may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7 (1993); 1^{st} Edition. If required the primer(s) may be labelled to facilitate detection.

The oligonucleotide primers and probes of the invention are particularly suitable for detecting the genotype of a particular polymorphism (e.g. SNP) of TDOA.

There are many conventional detectable labels such as radioisotopes, fluorescent labels, chemiluminescent compounds, labelled binding proteins, magnetic labels, spectroscopic markers and linked enzymes that might be used in conjunction with the primers or probes of the invention. One particular example well known in the art is end-labelling with ³²P. Fluorescent labels are preferred because they are less hazardous than radiolabels, they provide a strong signal with low background and various different fluorophors capable of absorbing light at different wavelengths and/or giving off different colour signals exists to enable comparative analysis in the same analysis. For example, fluorescein gives off a green colour, rhodamine gives off a red colour and both together give off a yellow colour.

Preferred primers for amplification are between 15 and 60 bp, more preferably between 17 and 35 bp in length. Probe sequences can be anything from about 25 nucleotides in length upwards. If the target sequence is a gene of 2 kb in size the probe sequence can be the complete gene sequence complement and thus may also be 2 kb in size. Preferably, the probe sequence is a genomic, or more preferably a cDNA, fragment of the target sequence and may be between 50 and 2000 bp, preferably between 200 and 750 bp. It will be appreciated that multiple probes each capable of selectively hybridising to a different target sequence of the TDOA nucleic acid, maybe across the complete length of the TDOA gene sequence, may be prepared and used together in a diagnostic test. The primers or probes may be completely homologous to the target sequence or may contain one or more mismatches to assist specificity in binding to the correct template sequence. Any sequence which is capable of selectively hybridising to the target sequence of interest may be used as a suitable primer or probe sequence. It will also be appreciated that the probe or primer sequences must hybridise to the target template nucleic acid. If the target nucleic acid is double stranded (genomic or cDNA) then the probe or primer sequence can hybridise to the sense or antisense strand. If however the target is mRNA (single stranded sense strand) the primer/probe sequence will have to be the antisense complement.

An example of a suitable hybridisation solution when a nucleic acid is immobilised on a nylon membrane and the probe nucleic acid is greater than 500 bases or base pairs is: 6×SSC (saline sodium citrate), 0.5% SDS (sodium dodecyl sulphate), 100 μg/ml denatured, sonicated salmon sperm DNA. The hybridisation being performed at 68° C. for at least 1 hour and the filters then washed at 68° C. in 1×SSC, or for higher stringency, 0.1×SSC/0.1% SDS.

An alternate example of “stringent hybridisation conditions” provides for an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulphate, and 20 pg/ml denatured, sheared salmon sperm DNA, followed by washing the filters in 0.1×SSC at 65° C. for at least an hour.

An example of a suitable hybridisation solution when a nucleic acid is immobilised on a nylon membrane and the probe is an oligonucleotide of between 12 and 50 bases is: 3M trimethylammonium chloride (TMACl), 0.01M sodium phosphate (pH 6.8), 1 mM EDTA (pH 7.6), 0.5% SDS, 100 μg/ml denatured, sonicated salmon sperm DNA and 0.1 dried skimmed milk. The optimal hybridisation temperature (Tm) is usually chosen to be 5° C. below the Ti of the hybrid chain. Ti is the irreversible melting temperature of the hybrid formed between the probe and its target. If there are any mismatches between the probe and the target, the Tm will be lower. As a general guide, the recommended hybridisation temperature for 17-mers in 3M TMACl is 48-50° C.; for 19-mers, it is 55-57° C.; and for 20-mers, it is 58-66° C.

Levels of TDOA gene expression can also be detected by screening for levels of polypeptide (TDOA protein). For example, monoclonal antibodies immunoreactive with TDOA protein can be used to screen a test sample. Such immunological assays can be done in any convenient format known in the art. These include Western blots, immunohistochemical assays and ELISA assays. Functional assays can also be used, such as protein binding determinations.

The TDOA protein of the invention and homologues or fragments thereof may be used to generate substances which selectively bind to it and in so doing regulate the activity of the protein. Such substances include, for example, antibodies, and the invention extends in particular to an antibody which is capable of binding to the protein shown in SEQ ID No:2. In particular the antibody may be a neutralising antibody.

As used herein the term antibody is to be understood to mean a whole antibody or a fragment thereof, for example a F(ab)₂, Fab, FV, VH or VK fragment, a single chain antibody, a multimeric monospecific antibody or fragment thereof, or a bi- or multi-specific antibody or fragment thereof. Each of these types of antibody derivative and their acronyms are well known to the person skilled in the art.

In another preferred embodiment antibodies directed against TDOA protein can be used, to detect, prognose, diagnose and stage OA disease. Various histological staining methods known in the art, including immunochemical staining methods, may also be used. Silver stain is but one method of detecting TDOA proteins. For other staining methods useful in the present invention see, for example, A Textbook of Histology, Eds. Bloom and Fawcett, W.B. Saunders Co., Philadelphia (1964).

According to a further aspect of the invention there is provided use of an antibody selective for TDOA protein, in an assay to diagnose or prognose or monitor OA.

The antibodies for use in this aspect of the invention can be prepared using the TDOA protein/polypeptides.

Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)). The term antibody includes both monoclonal antibodies, which are a substantially homogeneous population, and polyclonal antibodies, which are heterogeneous populations. The term also includes inter alia, humanised and chimeric antibodies. Monoclonal antibodies to specific antigens may be obtained by methods known to those skilled in the art, such as from hybridoma cells, phage display libraries or other methods. Monoclonal antibodies may be inter alia, human, rat or mouse derived. For the production of human monoclonal antibodies, hybridoma cells may be prepared by fusing spleen cells from an immunised animal, e.g. a mouse, with a tumour cell. Appropriately secreting hybridoma cells may thereafter be selected (Koehler & Milstein, Nature 256:495-497 (1975); Cole et al., “Monoclonal antibodies and Cancer Therapy”, Alan R Liss Inc, New York N.Y. pp 77-96 (1985)). Such antibodies may be of any immunoglobulin class including IgG, IgM, IgE, IgA, IgD and any subclass thereof.

Polyclonal antibodies can be generated by immunisation of an animal (such as a mouse, rat, goat, horse, sheep etc) with an antigen, such as one of the TDOA protein used in this invention.

The TDOA polypeptide(s) can be prepared by various techniques known to the person skilled in the art. RNA transcripts can be used to prepare a polypeptide of the invention by in vitro translation techniques according to known methods (Sambrook et al. supra). Alternatively, the TDOA polypeptide(s) can be synthesised chemically. For example, by the Merryfield technique (J. Amer. Chem. Soc. 85:2149-2154, 1968). Numerous automated polypeptide synthesisers, such as Applied Biosystems 431A Peptide Synthesizer also now exist. Alternatively, the TDOA polypeptide(s) are produced from a nucleotide sequence encoding the polypeptide using recombinant expression technology. A variety of expression vector/host systems may be used to express the TDOA coding sequences. These include, but are not limited to microorganisms such as bacteria expressed with plasmids, cosmids or bacteriophage; yeasts transformed with expression vectors; insect cell systems transfected with baculovirus expression systems; plant cell systems transfected with plant virus expression systems, such as cauliflower mosaic virus; or mammalian cell systems (for example those transfected with adenoviral vectors); selection of the most appropriate system is a matter of choice. Preferably, the TDOA protein is expressed in eukaryotic cells, especially mammalian, insect and yeast cells. Mammalian cells provide post-translational modifications to recombinant TDOA protein, which include folding and/or phosphorylation.

Expression vectors usually include an origin of replication, a promoter, a translation initiation site, optionally a signal peptide, a polyadenylation site, and a transcription termination site. These vectors also usually contain one or more antibiotic resistance marker gene(s) for selection. As noted above, suitable expression vectors may be plasmids, cosmids or viruses such as phage or retroviruses. The coding sequence of the polypeptide is placed under the control of an appropriate promoter, control elements and transcription terminator so that the nucleic acid sequence encoding the polypeptide is transcribed into RNA in the host cell transformed or transfected by the expression vector construct. The coding sequence may or may not contain a signal peptide or leader sequence for secretion of the polypeptide out of the host cell. Expression and purification of the TDOA polypeptide(s) can be easily performed using methods well known in the art (for example as described in Sambrook et al. supra).

The TDOA polypeptide(s), along with α-paxillin, can also be used in binding assays to identify compounds capable of inhibiting TDOA:α-paxillin binding.

The TDOA polypeptide(s) of the invention can also be used to inoculate animals, from which serum samples, containing the specific antibody against the introduced TDOA protein/polypeptide, can later be obtained.

Rodent antibodies may be humanised using recombinant DNA technology according to techniques known in the art. Alternatively, chimeric antibodies, single chain antibodies, Fab fragments may also be developed against the polypeptides of the invention (Huse et al., Science 256:1275-1281, 1989), using skills known in the art. Antibodies so produced have a number of uses, which will be evident to the molecular biologist or immunologist skilled in the art. Such uses include, but are not limited to, monitoring enzyme expression, development of assays to measure enzyme activity and use as a therapeutic agent. Enzyme linked immunosorbant assays (ELISAs) are well known in the art and would be particularly suitable for detecting the TDOA protein or polypeptide fragments thereof in a test sample.

The TDOA specific antibodies can be used in an ELISA assay to detect TDOA protein in body fluids or by immunohistochemistry or other means. In addition, an antibody could be used individually or as part of a panel of antibodies, together with a control antibody, which reacts to a common protein, on a dipstick or similar diagnostic device.

All the essential materials and reagents required for detecting TDOA in a test sample may be assembled together in a kit. Such a kit may comprise one or more diagnostic cDNA probes or oligonucleotide primers together with control probes/primers. The kit may contain probes immobilised on a microarray substrate such as a filter membrane or silicon-based substrate. The kit may also comprise samples of total RNA derived from tissues of various physiological states, such as normal, BPH, confined tumour and metastatic tumour, for example, to be used as controls. The kit may also comprise appropriate packaging and instructions for use in the methods of the invention.

According to another aspect of the present invention there is provided a diagnostic kit for diagnosing or prognosing or monitoring OA comprising, one or more diagnostic probe(s) and/or diagnostic primer(s) and/or antibodies capable of selectively hybridising or binding to TDOA.

It will be appreciated that the term “diagnostic kit” is not intended to limit the kit to diagnostic use only, it also encompasses other uses such as in prognostic, stage monitoring and therapeutic efficacy studies.

In a preferred embodiment, the diagnostic (detection) probes are provided on a microarray.

Such kits may further comprise appropriate buffer(s) and/or polymerase(s) such as thermostable polymerases, for example taq polymerase. They may also comprise companion/constant primers and/or control primers or probes. A companion/constant primer is one that is part of the pair of primers used to perform PCR. Such primer usually complements the template strand precisely. The kit may also contain control normal OA RNA labelled with one fluorophore (E.g. Cy5). In use, patient RNA derived from biopsy or body fluids or cells can be labelled with another fluorophore (e.g. Cy3), the RNAs could then be mixed and hybridised to the array. Instrumentation to detect fluorescence ratio e.g. of. Cy3:Cy5 are available and could be used to detect TDOA over-expression.

In another embodiment the kit comprises one or more specific probes suitable for hybridisation to mRNA in tissue sections in situ. The kit may also contain hybridisation buffer and detection reagents for colourimetric or fluorescence microscopy detection.

In another embodiment the kit comprises a set of specific oligonucleotide primers, optionally labelled, for quantitation by RT-PCR of TDOA mRNA. These primers may be Scorpion primers (Whitcombe et al., Nature Biotechnol. 17:804-807, 1999) allowing accurate quantitation of specific PCR product. Alternatively, Taqman or Molecular Beacon probes may be provided in the kit for this purpose. One form of the kit would be a microtitre plate containing specific reagents in several wells, to which aliquots of extracted RNA could be pipetted. The microtitre plate could be thermocycled on a suitable machine, which could also be capable of reading fluorescence emissions from plate wells (e.g. Perkin Elmer 7700).

In another embodiment the kit comprises one or more antibodies specific for the TDOA protein for use in immunohistochemical analysis.

In another embodiment the kit is an ELISA kit comprising one or more antibodies specific for the TDOA protein identified herein.

In another aspect of the invention there is provided a method for treating a patient suffering from OA comprising administering to said patient an effective amount of an antibody specific for TDOA.

According to another aspect of the invention, the TDOA gene may be used in gene therapy, for example where it is desired to modify the production of the protein in vivo, and the invention extends to such uses.

Knowledge of the gene according to the invention also provides the ability to regulate its expression in vivo by for example the use of antisense DNA or RNA. One therapeutic means of inhibiting or dampening the expression levels of a particular gene (for example TDOA identified herein) is to use antisense therapy. Antisense therapy utilises antisense nucleic acid molecules that are synthetic segments of DNA or RNA (“oligonucleotides”), designed to mirror specific mRNA sequences and block protein production. Once formed, the mRNA binds to a ribosome, the cell's protein production “factory” which effectively reads the RNA sequence and manufactures the specific protein molecule dictated by the gene. If an antisense molecule is delivered to the cell (for example as native oligonucleotide or via a suitable antisense expression vector), it binds to the messenger RNA because its sequence is designed to be a complement of the target sequence of bases. Once the two strands bind, the mRNA can no longer dictate the manufacture of the encoded protein by the ribosome and is rapidly broken down by the cell's enzymes, thereby freeing the antisense oligonucleotide to seek and disable another identical messenger strand of mRNA.

Thus, according to another aspect of the invention there is provided a method for treating a patient suffering from OA comprising administering to said patient an effective amount of an anti-sense molecule capable of binding to the mRNA of the TDOA gene, and inhibiting expression of the protein product of the TDOA gene.

Complete inhibition of protein production is not essential, indeed may be detrimental. It is likely that inhibition to a state similar to that in normal tissues would be desired.

This aspect of antisense therapy is particularly applicable if the OA disorder is a direct cause of over-expression of the TDOA gene in question, although it is equally applicable if said TDOA gene indirectly cause the OA disorder. With knowledge of the TDOA gene and mRNA sequence, the person skilled in the art is able to design suitable antisense nucleic acid therapeutic molecules and administer them as required.

Antisense oligonucleotide molecules with therapeutic potential can be determined experimentally using well-established techniques. To enable methods of down-regulating expression of the TDOA gene of the present invention in mammalian cells, an example antisense expression construct can be readily constructed for instance using the pREP10 vector (Invitrogen Corporation). Transcripts are expected to inhibit translation of the gene in cells transfected with this type of construct. Antisense transcripts are effective for inhibiting translation of the native gene transcript, and capable of inducing the effects (e.g., regulation of tissue physiology) herein described. Oligonucleotides which are complementary to and hybridisable with any portion of TDOA gene mRNA are contemplated for therapeutic use. U.S. Pat. No. 5,639,595, “Identification of Novel Drugs and Reagents”, issued Jun. 17, 1997, wherein methods of identifying oligonucleotide sequences that display in vivo activity are thoroughly described, is herein incorporated by reference. Expression vectors containing random oligonucleotide sequences derived from the TDOA gene sequence are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material. Antisense molecules can be synthesised for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, “Hybrid Oligonucleotide Phosphorothioates”, issued Jul. 29, 1997, and U.S. Pat. No. 5,652,356, “Inverted Chimeric and Hybrid Oligonucleotides”, issued Jul. 29, 1997, which describe the synthesis and effect of physiologically-stable antisense molecules, are incorporated by reference. Antisense molecules may be introduced into cells by microinjection, liposome encapsulation or by expression from vectors harboring the antisense sequence.

As noted above, antisense nucleic acid molecules may also be provided as RNAs, as some stable forms of RNA are now known in the art with a long half-life that may be administered directly, without the use of a vector. In addition, DNA constructs may be delivered to cells by liposomes, receptor mediated transfection and other methods known to the art.

The antisense DNA or RNA for co-operation with the gene in SEQ ID No:1 can be produced using conventional means, by standard molecular biology and/or by chemical synthesis as described above. If desired, the antisense DNA or antisense RNA may be chemically modified so as to prevent degradation in vivo or to facilitate passage through a cell membrane and/or a substance capable of inactivating mRNA, for example ribozyme, may be linked thereto and the invention extends to such constructs.

The antisense DNA or antisense RNA may be of use in the treatment of diseases or disorders in humans in which the over- or under-regulated production of the TDOA gene product has been implicated.

Alternatively, ribozyme molecules may be designed to cleave and destroy the TDOA mRNA in vivo. Ribozymes are RNA molecules that possess highly specific endoribonucleases activity. Hammerhead ribozymes comprise a hybridising region, which is complementary in nucleotide sequence to at least part of the target RNA, and a catalytic region, which is adapted to recognise and cleave the target RNA. The hybridising region preferably contains at least 9 nucleotides. The design, construction and use of such ribozymes is well known in the art and is more fully described in Haselhoff and Gerlach, (Nature. 334:585-591, 1988). In another alternative oligonucleotides designed to hybridise to the 5′-region of the TDOA gene so as to form triple helix structures may be used to block or reduce transcription of the TDOA gene. In another alternative, RNA interference (RNAi) oligonucleotides or short (18-25 bp) RNAi TDOA sequences cloned into plasmid vectors are designed to introduce double stranded RNA into mammalian cells to inhibit and/or result in the degradation of TDOA messenger RNA. TDOA RNAi molecules may begin adenine/adenine (AA) or at least (any base-A,U,C or G)A . . . and may comprise of 18 or 19 or 20 or 21 or 22 or 23, or 24 or 25 base pair double stranded RNA molecules with the preferred length being 21 base pairs and be specific to individual TDOA sequences with 2 nucleotide 3′ overhangs or hairpin forming 45-50mer RNA molecules. The design, construction and use of such small inhibitory RNA molecules is well known in the art and is more fully described in the following: Elbashir et al., (Nature. 411(6836):494-498, 2001); Elbashir et al., (Genes & Dev. 15:188-200, 2001); Harborth, J. et al. (J. Cell Science 114:4557-4565, 2001); Masters et al. (Proc. Natl. Acad. Sci. USA 98:8012-8017, 2001); and, Tuschl et al., (Genes & Dev. 13:3191-3197, 1999).

Pathway mapping may be used to determine each protein in the cell with which the TDOA protein interacts and, in turn, the proteins with which each of these proteins interacts also. In this way it is possible to identify the specific critical signalling pathway which links the disease stimulus to the cell's response thereby enabling the identification of new potential targets for therapy intervention. As outlined in Examples, particularly Examples 5 and 7, the inventors have identified that TDOA interacts with α-paxillin.

According to a further aspect of the invention there is provided the use of the TDOA gene or a fragment thereof in research to identify further gene targets implicated in OA.

According to a further aspect of the invention there is provided a method of treating a human in need of treatment with a small molecule drug acting on the TDOA protein or an anti-sense oligonucleotide acting against the TDOA mRNA, in which the method comprises:

i) detection of a polymorphism in the TDOA gene in the human, which diagnosis preferably comprises determining the sequence at one or more positions selected from the group consisting of: positions 30983, 38160, 12523 . . . 12542 and 32945 of the TDOA gene (as defined by the position in SEQ ID NO: 1), or a polymorphism in linkage disequilibrium above D′ 0.9 therewith;

ii) determining the status of the human by reference to polymorphism in the TDOA gene; and,

iii) administering an effective amount of the drug.

In another embodiment, this aspect is applied to the detection or one or more of the polymorphisms identified in Table 2.

According to a further aspect of the invention there is provided a method of treating a human in need of treatment with a small molecule drug acting on the TDOA protein or an anti-sense oligonucleotide acting against the TDOA mRNA, in which the method comprises:

i) measuring the level of the TDOA mRNA in a tissue sample obtained from the human and,

ii) determining the status of the human by reference to normal levels of the TDOA mRNA; and,

iii) administering an effective amount of the drug.

According to a further aspect of the invention there is provided a method of treating a human in need of treatment with a small molecule drug acting on the TDOA protein or an anti-sense oligonucleotide acting against the TDOA mRNA, in which the method comprises:

i) measuring the level of the TDOA protein in a tissue sample obtained from the human and,

ii) determining the status of the human by reference to normal levels of the TDOA protein; and,

iii) administering an effective amount of the drug. Included within the group of compounds that act on the TDOA protein are those that can either disrupt or inhibit the interaction between TDOA and α-paxillin or TDOA and itself (i.e. form multimers).

According to another aspect of the invention there is provided a method of treatment of a patient suffering from an OA disorder, comprising administration to the patient of a compound capable of reducing the transcription or expression of TDOA.

According to another aspect of the invention there is provided a method of treatment of a patient suffering from an OA disorder, comprising administration to the patient of a compound capable of inhibiting the binding of TDOA to α-paxillin or to itself.

According to another aspect of the invention there is provided a method of treatment of a patient suffering from an OA disorder, comprising administration to the patient an antisense nucleic acid molecule targeted against the mRNA of TDOA.

According to another aspect of the invention there is provided the use of an antisense nucleic acid molecule or an antibody directed against TDOA, in the manufacture of a medicament for treating an OA disorder.

Each aspect of the invention is not only applicable to OA, including primary and secondary OA but others arthropathies, such as intervertebral disc degeneration and temporomandibular joint disease; metabolic arthropies such as chronic crystal deposition disease including CPPD and hydroxyapatite as well as contributing to inflammatory disease such as rheumatoid arthritis.

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook and Russell, Molecular Cloning: A Laboratory Manual, 3d ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

The invention will be further described by way of the following non-limiting examples and figures in which,

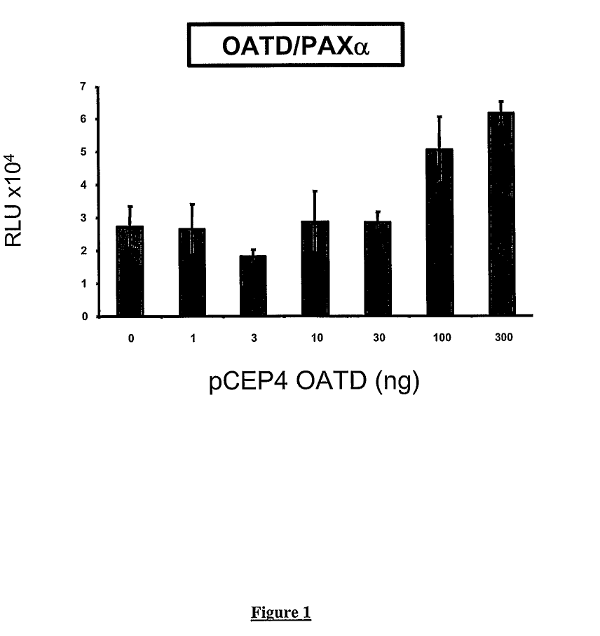

FIG. 1 is a bar chart demonstrating that expression of TDOA increased the signal of binding between TDOA and α-paxillin in mammalian 2-hybrid; and

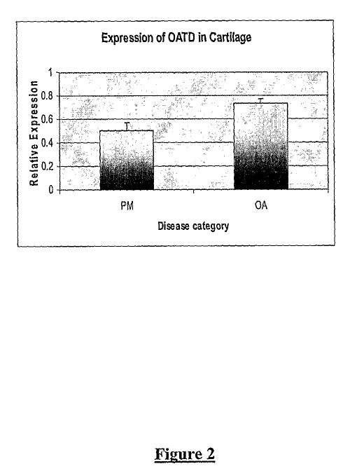

FIG. 2 is a bar chart showing expression levels of TDOA mRNA in cartilage from normal post-mortem samples (PM) and OA patients (OA). Samples include RNA derived from medial and lateral femoral condyles and tibal plateaus. TDOA expression was normalised to 18S RNA.

TDOA is also referred to herein as OATD.

### EXAMPLE 1

### Identification of TDOA as a Gene Associated with OA

We investigated the 11q region of interest using a linkage-based approach based on SNP genotyping.

### 1. Methods

The 20 cM 11q region described in WO 00/20632, is delimited by the microsatellite markers D11S4046 and D11S1320. Linkage disequilibrium analysis identified excess transmission of the 258 bp allele of marker D11S937 in cases (i.e. patients diagnosed with OA who had undergone joint replacement) compared to controls (e.g. asymptomatic spouses of patients).

In order to finely map of this region we performed SNP discovery and genotyping to decrease the inter-marker distance. Genotyping was carried out on 600 DNAs from subjects suffering from OA of the knee or hip and who had undergone joint replacement. Genotyping of 500 control DNAs from the asymptomatic spouses of the cases was also performed.

### A. SNP Discovery

SNPs occurring between the markers D11S4046 and D11S1320 were identified by the following methods.

### (i) BAC (Bacterial Artificial Chromosome) Shotgun Library Screening

Hybridisation against the deJong RCPI-11 BAC library (Osoegawa et al. Genome Research. 11 (3):483-496, 2001) was undertaken with pools of ³²P end labelled oligos derived from STSs (Sequence Tagged Sites) in the region of interest. BACs were then checked individually by PCR and 137 individual BACs were isolated.

BAC DNA was prepared from 500 ml overnight cultures using an extraction kit (Nucleon/Tepnel, Manchester UK). Two methods were used to construct libraries. In the first method, BAC DNA was partially digested with Sau3a and separated by agarose gel electrophoresis. Fragments in the 0.5-4 kb size range were cut out of the gel and purified. The fragments were shotgun cloned into the pZERO plasmid vector (Invitrogen) and single colonies were picked and the inserts were PCR amplified using conventional T7 and SP6 primers. PCR products were sequenced using dye terminator chemistry. In the second method, BAC DNA was sheared to 1 Kb in size using a Hydroshear (GeneMachines, San Carlos, Calif.) and sub-cloned into the pZERO plasmid vector. Single colonies were picked and the inserts were PCR amplified using conventional M13 forward and reverse primers. PCR products were sequenced on the MegaBace (Amersham Pharmacia, Little Chalfont, UK) using M13 F&R primers.

Sequences generated from the PCR products in all libraries were assembled into a contig using the SeqMan package (DNAStar, Madison Wis.). Sequences over 500 bp in size were analysed, trimmed for vector and quality and masked for repeats/E. coli DNA and novel BAC sequence was identified. Primers were designed to this sequence to generate 400-600 bp PCR products for use in the polymorphism screening protocols. A total of 837 STSs were generated for screening. All primer pairs were tested in 3 separate amplification conditions and working primers were put through a polymorphism screen in 1 pool (17 individuals) and 3 individuals were sequenced in the forward and reverse direction. Analysis of the sequences was done using Phred/Phrap/Consed.

Analysis of the shotgun libraries generated a total of 132 SNPs. An average of 1 SNP was discovered for every 1.6 kb sequence analysed.

### (ii) Analysis of Genomic Clones

We identified 330 genomic clones (Research Genetics) mapping to the region of interest and were able to use the sequences directly to provide sequence for polymorphism screening. The Genomic clone sequence was used to design at least 6 primer pairs for each clone and subsequent screening was performed by a combination of WAVE (Transgenomic Inc. Crewe, UK) and sequence analysis.

141 SNPs were discovered by this method.

(iii) The SNP Consortium SNPs

The fourth release of The SNP Consortium's database of human single nucleotide polymorphisms contained 102,719 SNPs, all of which were anchored to the human genome by radiation hybrid mapping and/or “in silico” mapping to the genomic sequence working draft.

A total of 168 uniquely represented SNPs were retrieved from the 20 Mb region of interest.

### B. SNP Genotyping

The SNPs discovered in Step A were genotyped in 600 DNAs from subjects suffering from OA of the knee or hip and who had undergone joint replacement and 500 control DNAs from the asymptomatic spouses of the cases. The genotyping was carried out using allele specific amplification (ARMS) and single nucleotide primer extension.

### C. Microsatelite Genotyping

Ten microsatellite markers, D11S916, D11S1321, D11S4081, D11S4128, D11S4179, D11S911, D11S4079, D11S906, D11S937, D11S4166, were genotyped in the case control cohorts.

### EXAMPLE 2

### Linkage Analysis

### A. Microsatellites

Analysis of the microsatellites was carried out in the program CLUMP (author D. Curtis, Human Genome Mapping Project). This program compares the distribution of alleles across cases and controls generating a single p value, thus negating the need for correction of multiple tests for each allele (not each marker). The program also carries out monte-carlo simulations, producing an exact p value for the data.

The association was confirmed for D11S937 (p=0.04) and a further association was seen with D11S4081 (p 0.02), which maps approximately 0.7 cM from D11S937.

### B. SNPs

Allele frequencies were compared for each SNP between cases and controls, ODDs ratios were calculated with 95% confidence intervals (Jurg Ott, Analysis of Human Genetic Linkage 3^{rd} Ed, John Hopkins Univ. Press, 1999). Analysis of SNPs with an allele frequency of >5% which were in Hardy-Weinberg equilibrium revealed three (SEQ ID Nos: 3-5 & Table 3) which showed significant association with the disease (p. <0.05). These were not corrected for multiple testing. It was found that all three SNPs lay within the same 3 cM radiation hybrid interval (termed interval D), which also mapped close to microsatelite marker D11S937 located at 11q13.5.

### EXAMPLE 3

### Gene Discovery

35 Bacterial Artificial Chromosomes (BACs) were found to map to interval D. Following analysis using Seqman. The BACs were assembled into 138 contigs covering 2.8 Mb. The gene termed TDOA was found within this interval.

### EXAMPLE 4

### SNP Discovery

Following publication of the Ensemble sequence for this region the cDNA for TDOA was mapped to the genomic sequence to identify the exon/intron boundaries, the UTRs and potential promotor areas.

PCR primers were designed to span the exons, UTRs, potential promotor areas and the flanking intronic sequence. Sequencing was carried out in 29 unrelated Caucasian individuals and the sequence was aligned in Polyphred/Phrap/Consed to identify polymorphisms.

The polymorphisms discovered are shown in Table 2.

TABLE 2
FEATURES OF TDOA GENE AND FLANKING GENOMIC
SEQUENCE LOCATION is by nucleotide number and
refers to SEQ ID NO: 1

| LOCATION | FEATURE TYPE | FEATURE ID |
|---|---|---|
| 2250 | SNP | OA_74 |
| 3098 | SNP | OA_75 |
| 3201 | SNP | OA_76 |
| 3768 | SNP | OA_77 |
| 4021 | SNP | OA_78 |
| 4349 | SNP | OA_79 |
| 5538 | SNP | OA_80 |
| 7877 | SNP | OA_81 |
| 8588 | SNP | OA_82 |
| 9243 | SNP | OA_83 |
| 9274 | SNP | OA_84 |
| 9413 | SNP | OA_85 |
| 10564 | SNP | OA_86 |
| 11868 | SNP | OA_87 |
| 12024 | SNP | OA_88 |
| 12803 | SNP | OA_89 |
| 12855 | SNP | OA_90 |
| 13425 | SNP | OA_91 |
| 13781 | SNP | OA_92 |
| 13806 | SNP | OA_93 |
| 19626 | SNP | OA_94 |
| 20661 | SNP | OA_95 |
| 20696 | SNP | OA_96 |
| 20721 | SNP | OA_97 |
| 21138 | SNP | OA_98 |
| 21607 | SNP | OA_99 |
| 23231 | SNP | OA_101 |
| 23446 | SNP | OA_102 |
| 24334 | SNP | OA_103 |
| 25214 | SNP | OA_104 |
| 25406 | SNP | OA_105 |
| 25929 | SNP | OA_106 |
| 27069 | SNP | OA_107 |
| 27503 | SNP | OA_108 |
| 27794 | SNP | OA_109 |
| 28363 | SNP | OA_110 |
| 28462 | SNP | OA_111 |
| 28561 | SNP | OA_112 |
| 28741 | SNP | OA_113 |
| 28955 | SNP | OA_114 |
| 29059 | SNP | OA_115 |
| 29821 | SNP | OA_116 |
| 30250 | SNP | OA_117 |
| 30700 | SNP | OA_118 |
| 30760 | SNP | OA_119 |
| 30983 | SNP | OA_120 |
| 31238 | SNP | OA_121 |
| 31280 | SNP | OA_122 |
| 31335 | SNP | OA_123 |
| 31377 | SNP | OA_124 |
| 31712 | SNP | OA_125 |
| 32195 | SNP | OA_126 |
| 32639 | SNP | OA_127 |
| 32945 | SNP | OA_128 |
| 33272 | SNP | OA_129 |
| 33540 | SNP | OA_130 |
| 34219 | SNP | OA_131 |
| 34641 | SNP | OA_132 |
| 35074 | SNP | OA_133 |
| 36601 | SNP | OA_134 |
| 37131 | SNP | OA_135 |
| 37525 | SNP | OA_136 |
| 37556 | SNP | OA_137 |
| 38160 | SNP | OA_138 |
| 38586 | SNP | OA_139 |
| 38731 | SNP | OA_140 |
| 39608 | SNP | OA_141 |
| 39887 | SNP | OA_142 |
| 40087 | SNP | OA_143 |
| 40409 | SNP | OA_144 |
| 40501 | SNP | OA_145 |
| 40879 | SNP | OA_146 |
| 46006 | SNP | OA_147 |
| 48083 | SNP | OA_148 |
| 49053 | SNP | OA_149 |
| 49144 | SNP | OA_150 |
| 49169 | SNP | OA_151 |
| 2208 . . . 2249 | Microsatellite | D11S937 |
| 12523 . . . 12542 | 20bp INDEL | 0A_100 |
| 30152 . . . 32674 | 3′ UTR | exon4 |
| 32675 . . . 33457 | CODING EXON | exon3 |
| 33458 . . . 33488 | 5′ UTR | exon2 |
| 47477 . . . 47521 | 5′ UTR | exon1 |

TABLE 3
Polymorphisms showing positive association to OA
(ODDs ratio with p value < 0.05)

| | | | Allele | |
|---|---|---|---|---|
| FEA- | | | showing | |
| TURE | location within | Nucleotide | association | |
| ID | SEQ ID NO: 1 | Change | with OA | Sequence ID |
| OA_120 | 30983 | G to A | A | SEQ ID No: 4 |
| OA_138 | 38160 | A to G | G | SEQ ID No: 5 |
| OA_100 | 12523 . . . 12542 | Del to Ins | Del | SEQ ID No: 6 |

SEQ ID Nos: 4, 5 and 6 depict the associated polymorphism with adjacent sequence to facilitate locating the polymorphism in other variant TDOA sequences.

TABLE 4
SNPs that occur within the amino acid coding region of the TDOA gene.
Locations are by amino acid residue number and refer to SEQ ID NO: 2.
SNP IDs refer to those listed in Table 2.

| Location | SNP ID | Nucleotide Change | Conserved Amino acid |
|---|---|---|---|
| 170 | OA_128 | C to A | Leucine |

### EXAMPLE 5

### The link with α-Paxillin

TDOA had no known functional link to OA and no reported biological function within the academic literature. TDOA is identical to TRICH-6 found in the GeneSeqP patent database (accession number: ABG61536). Based on in silico homology alignment analyses, TDOA was found to possess weak similarity to potassium ion channels due to its homology with the Ti domain, however, TDOA does not have an obvious ion transport domain.

In order to understand the possible function of TDOA we undertook a pathway analysis experiment using yeast 2-hybrid to look for proteins that associate with TDOA.

The Matchmaker GAL4 Two-Hybrid System 3 (Clontech) was used to screen for protein interaction partners of TDOA. In the Matchmaker 3 System TDOA was expressed as a “bait” fused to the DNA binding domain of the transcription factor Gal4 and potential interacting partners for TDOA (“prey”) was co-expressed in the yeast as a fusion with the transactivation domain of Gal4.

Interaction between the TDOA “bait” and binding partner “prey” reconstitutes the transcriptional activity of Gal4 resulting in expression of a reporter gene under the control of the Gal4 promoter. Expression of this gene can be use for detection and selection of yeast expressing the “prey”. The construct containing the “prey” can be isolated from the yeast and the “prey” sequenced and identified.

The TDOA “bait” was constructed by insertion of the TDOA cDNA (SEQ ID NO: 3) into the vector pGBKT7. A cDNA library (“prey”) was prepared from Human Spinal Cord by oligo dT priming, and directionally cloned into 5′EcoRI-XhoI of pACT2-AD (Clontech). Titre of the library was 6.8×10⁴ colonies/ml. Primary recombinants were estimated to be 4.1×10⁶ total clones. The insert size was from 0.9-3.7 kb with an average size (10 clones) of 1.82 kb. 1.2×10⁶ clones were screened in strain Ah109 under high stringency conditions, whereby the transformants are required to grow on SD (synthetic drop out media)-Trp, -Leu, -His, -Ade and be positive for lacZ, i.e. blue in the presence of X-α-gal substrate.

The screen identified two cDNA hits that were sequenced and shown to contain 541 (clone B2; SEQ ID NO: 10) and 664 (clone B1; SEQ ID NO: 11) base pair fragments of human α-paxillin. The two clones were overlapping and showed 100% identity with a central region of α-paxillin. As the other known splice variants of paxillin (β-paxillin and γ-paxillin) both contain inserts within this region, it was clear that these clones were of α-paxillin.

The interaction between TDOA and α-paxillin identified by yeast 2-hybrid was confirmed by immunoprecipitation/Western blot analysis.

In order to understand the function of TDOA we examined the effects of TDOA overexpression on other cellular proteins using 2D Difference Gel Electrophoresis.

HEK 293T cells were stably transfected using Retrovirus as follows: cDNA clones encoding either TDOA (SEQ ID NO: 3) or TDOA containing a C-terminal FLAG tag (SEQ ID NO: 7) were cloned into the Gateway® (Invitrogen) adapted vector pFUNC-GW using standard Gateway® cloning methodologies. This yielded two retroviral expression vectors; pFUNC1-GW untagged TDOA and pFUNC1-GW 5′FLAG TDOA. The destination vector was linearised with Not I (New England Biolabs) and untagged and 5′flag tagged TDOA (SEQ ID NO: 7) were cloned by performing an LR recombination reaction using the LR Clonase kit (Invitrogen). After 16 hours at 16° C., the recombination reactions were treated with proteinase K (Invitrogen) for 10 min at 37° C., and then 1 ml was transformed using DH5a Max Efficiency competent cells (Invitrogen) and plated onto L+ampicillin agar plates.

### Retroviral Generation

Replication deficient retroviral particles capable of expressing either untagged or C-terminally FLAG tagged TDOA were pseudotyped with vesicular stomatitis virus G protein (VSV-G) coat protein by transient transfection of HEK 293T cells. Twenty-four hours prior to transfection, HEK 293T cells (3×10⁶) were plated onto 10-cm tissue culture dishes. Using the calcium phosphate-mediated ProFection® Mammalian Transfection System (Promega), cells were co-transfected with three vectors: the gag-pol expression vector pVPack-GP (Stratagene), the VSV-G envelope expression vector pVPack-VSV-G (Stratagene), and the appropriate retroviral expression vector containing the cDNA of interest (either pFUNC1-GW untagged TDOA or pFUNC1-GW 5′FLAG TDOA). Sixteen micrograms of the appropriate retroviral expression vector, 16 μg of pVPack-GP and 8 μg of pVPack-VSV-G were combined with 62.5 μl of 2 M calcium chloride to a volume of 500 μl. This solution was then added drop wise to 500 μl of 2′ HEPES-buffered saline, with constant aeration through a mechanical pipetter. After 15 min of incubation at room temperature, the DNA precipitate was distributed evenly over the cells. The cells were then incubated overnight at 37° C. The next day, medium was replaced with fresh growth medium supplemented with 10 mM sodium butyrate, in order to enhance transcription from the transfected vectors (18). After 6 h of incubation at 37° C., medium was replaced again, without sodium butyrate, and the cells were incubated overnight at 37° C. for generation of the retroviral supernatant. The supernatant was harvested 48 h post-transfection, filtered through a 0.45-μm filter, and either was used immediately for infections or was frozen on dry ice, stored at −80° C. and used in later infections.

### HEK 293T Transductions

Twenty-four hours prior to infection, HEK 293T cells (1.5×10⁵) were plated onto 60-mm tissue culture dishes. The cells were infected with retroviral supernatant supplemented with 5 μg/ml polybrene, and then incubated overnight at 37° C. The next day, medium and retroviral supernatant were removed, and the cells transferred to fresh growth medium. Forty-eight hours post-infection, cells were split from the 60-mm tissue culture dishes into 75-cm2 flasks, and, after allowing cells to adhere, medium was supplemented with 0.5 μg/ml puromycin to select cells transduced with proviruses. In general, puromycin selection continued for two weeks until clonal colonies became established, after which the cells were expanded and maintained as for the other cell lines.

For analysis of the infection efficiency of the retroviral supernatant, infections were performed as described above, except using HEK 293T cells in 6-well plates and without puromycin selection

Two cell lines expressing either untagged TDOA (HEK-untagged-TDOA) or C-terminally FLAG tagged TDOA (HEK-C-FLAG-TDOA) were generated.

Cell lysates were then prepared from HEK 293T cells expressing either untagged TDOA (HEK-untagged-TDOA) or FLAG-tagged TDOA (HEK-C-FLAG_TDOA) as follows: cells were resuspended at 5×10⁷ cells/ml in lysis buffer (Tris buffered saline (TBS)/1% IGEPAL CA-630+ protease/phosphatase inhibitors: 150 mM NaCl, 50 mM Tris, pH 8, 1% IGEPAL CA-630, 10 mM NaF, 2 mM Na₃VO₄, 10 mM β-glycerophosphate, 1×EDTA-free Complete™ protease inhibitor tablet per 50 ml buffer (Roche Applied-Science), 1 mM DTT) and the samples vortexed. Cells were lysed by incubating on ice for 30 min with occasional mixing. The lysates were centrifuged at 100 000 g for 30 min at 4° C. The supernatants were removed and used for immunoprecipitations

Anti-FLAG immunoprecipitations (IPs) were performed from HEK-untagged-TDOA and HEK-C-FLAG-TDOA cell lysates using Ultralink-hydrazide beads (Pierce) conjugated to anti-FLAG M2 monoclonal antibodies (Sigma). Antibody conjugation was performed essentially as described by the manufacturer.

Approximately 25 μl of anti-FLAG-conjugated Ultralink-hydrazide beads were use to immunoprecipitate FLAG tagged TDOA from HEK 293 cell lysates equivalent to approximately 10⁷ cells as follows: ensuring the sample/bead slurry was well-mixed, 500 μl was transferred to a Handee spin cup (Pierce) and centrifuged at 14 000 rpm in a microcentrifuge for 2 min. The flow through was decanted into a clean tube. The remaining sample/bead slurry was well mixed and transferred to the same spin cup, centrifuging for 2 min until all the beads were collected in the same spin cup.

The beads were washed for 5 min by resuspending in 500 μl TBS/1% IGEPAL on a rotary mixer at 4° C. Samples were centrifuged for 2 min to remove each wash into a clean Handee microcentrifuge tube (Pierce). This was repeated a further 3 times. Finally beads were washed for 5 min with 50 μl TBS/1% IGEPAL on a vibromat at 4° C. Samples were centrifuged for 2 min to remove the final wash. Protein was eluted from the beads by the addition of 50 μl of 0.4 mg/ml FLAG peptide diluted in TBS/1% IGEPAL for 30 min at 4° C. on a vibromat. Samples were centrifuged for 2 min to collect each eluate.

Cell lysate, IP flow through and IP eluate samples were separated on Novex Tris-glycine 8-16% acrylamide gels (Invitrogen) as per the manufacturer's instructions. Proteins were then transferred to PVDF membrane using Novex blotting units (Invitrogen) following the manufacturer's instructions. The gels were blotted for 1.5 hr at 200 mA. The blots were then blocked overnight at 4° C. with blocking buffer (Phosphate Buffered Saline (PBS; 0.01M phosphate buffer, 0.0027M potassium chloride. pH7.4), 0.1% (v/v) Tween 20 and 5% (w/v) non-fat dried milk powder).

The blots were washed 2×5 min in wash buffer (PBS, 0.1% (v/v) Tween 20 and 0.5% (w/v) non-fat dried milk powder) and then incubated for 2.5 hr at room temperature with anti-paxillin antibodies (Anti-Paxillin Mouse monoclonal IgG1, Upstate, code: 05-417) diluted in wash buffer to the supplier's recommendation.

The blots were then washed 3×5 min in wash buffer and incubated for 2.5 hr with secondary antibody detection antibody (goat anti-mouse IgG (H+L), horseradish peroxidase conjugate—Invitrogen) in wash buffer diluted to the supplier's recommendation. The blots were then washed 2×10 min in wash buffer then 2×10 min in PBS. Antibody signal was detected using SuperSignal West Pico enhanced chemiluminescent substrate according to the manufacturers instructions. The blots were then blotted dry, placed between 2 acetates and exposed to Hyperfilm.

A protein band corresponding to α-paxillin was detected immunoprecipitating with TDOA. This confirmed the interaction between TDOA and α-paxillin detected by Y2H.

### EXAMPLE 6

In order to analyse the effects of TDOA overexpression on cellular protein expression, untransfected HEK EBNA cells and HEK EBNA cells that had been stably transfected with untagged TDOA using retrovirus (as above) were seeded into 225 cm² flasks and grown as monolayers for 5 days and then harvested using mild trypsinisation. 50 μg loadings of either Cy5-labelled untransfected or Cy5-labelled TDOA-expressing HEK EBNA cell lysate were combined with 50 μg Cy3-labelled pooled untransfected and TDOA-expressing HEK EBNA lysate (internal standard). pH 4-7 and pH6-11 1^{st} dimension separations carried out followed by 12.5% acrylamide 2^{nd} dimension separations. Gels were imaged at wavelengths for Cy3 and Cy5 (2 images/gel). Image analysis was carried out using Progenesis Discovery. Spot filter was applied to select spots showing a greater than 2-fold change in expression between the control and TDOA-expressing samples, with a t-test threshold of <0.05. Silver and Colloidal Coomassie gels of 1 mg loadings were performed for spot cutting and MS analysis (MALDI).

A total of 10 protein spots were identified as having changed significantly following TDOA overexpression, of which 5 were positively identified (Table 5).

TABLE 5
MALDI-MS analysis results. A positive fold change indicates a protein with
increased expression in the TDOA-expressing cells, a negative fold change indicates
a protein with decreased expression in the TDOA-expressing cells.

| Gel | | | | |
|---|---|---|---|---|
| pH | Gene | Gene Description | Fold Change | Significance |
| 4-7 | PP1B_HUMAN | Ser/thr protein phosphatase | −3.779 | p = 0.0447 |
| | | PP1_beta catalytic subunit | | |
| 4-7 | ROK_HUMAN | Hetergeneous nuclear | 2.09 | p = 0.0105 |
| | | ribonuclearprotein | | |
| 6-11 | EF2_HUMAN | Elongation factor 2 | −3.496 | p = 0.00049 |
| 4-7 | AAE10329 | Human transporter and ion- | 11.4 | p = 5.751e−008 |
| | | channel-6 (TRICH-6) | | |
| 6-11 | AAE10329 | Human transporter and ion- | 11.267 | p = 4.652e−006 |
| | | channel-6 (TRICH-6) | | |

### Literature Analysis of TDOA Interactors.

TABLE 6
Protein changes from 2D gels:

| | | Fold | |
|---|---|---|---|
| Gene | Gene Description | Change | Significance |
| PP1B_HUMAN | Ser/thr protein | −3.779 | p = 0.0447 |
| | phosphatase PP1_beta | | |
| | catalytic subunit | | |
| ROK_HUMAN | Hetergeneous nuclear | 2.09 | p = 0.0105 |
| | ribonuclearprotein | | |
| EF2_HUMAN | Elongation factor 2 | −3.496 | p = 0.00049 |
| AAE10329 | Human transporter and | 11.4 | p = 5.751e−008 |
| | ion-channel-6 (TRICH-6) | | |
| AAE10329 | Human transporter and | 11.267 | p = 4.652e−006 |
| | ion-channel-6 (TRICH-6) | | |

TABLE 7
Data from an in-house OA patient versus post mortem cadaver (PM)
articular cartilage Affymetrix experiment demonstrated the following
gene expression changes for PP1B and EEF2 in articular cartilage
samples from OA donors:

| Gene | Gene Description | Fold Change | Significance |
|---|---|---|---|
| PP1B_HUMAN | Ser/thr protein | −2.08 | p = 0.0019 |
| | phosphatase PP1_beta | | |
| | catalytic subunit | | |
| ROK_HUMAN | Hetergeneous nuclear | 1.1 | p = 1.0 |
| | ribonuclearprotein | | |
| EF2_HUMAN | Elongation factor 2 | −1.72 | p = 0.000126 |

In summarise, the expression of three proteins (PP1 CB, hnRPK and EEF2) was altered by overexpression of TDOA. PP1 CB and EEF2 were similarly changed in OA cartilage at the genomic level. A review of the functionality of these proteins has suggested possible roles for them in OA.

### EXAMPLE 7

The interaction between TDOA and α-paxillin was further confirmed in mammalian 2-hybrid experiments using the CheckMate™ system (Promega). The protocol was essentially as described by the manufacturer. Briefly, TDOA and α-paxillin (cDNA sequence disclosed as SEQ ID NO: 12) were cloned into either the DNA binding domain construct (pBind) or transactivation domain (pAct) construct. 5×10⁴ HEK293T cells in 100 μl of growth medium (DMEM plus glutamax (Invitrogen, Paisley, UK) supplemented with 10% foetal calf serum (FCS) (Hyclone, Cramlington, UK) and 1% penicillin/streptomycin (Invitrogen)) were added to each well of a 96 well plate (poly-D-Lysine treated, black/clear well, cat# 356640 Becton Dickinson, Oxford, UK). These cells were grown overnight to 85-95% confluency and transfected the next day.

The medium on the cells to be transfected was changed to antibiotic free medium (DMEM+glutamax with 10% FCS) before further treatment. 1 μl of lipofectamine 2000 (Invitrogen) was added to 25 μl of Optimem tissue culture medium (Invitrogen) and incubated for 5 mins. Approximately 10 ng of each plasmid to be transfected (at a molar ratio) was added to a separate aliquot of 25 μl Optimem. These two aliquots of Optimem were mixed and incubated at room temperature for 20 mins, then added to the cells and incubated overnight.

Post-transfection the medium was removed from the cells and 25 μl of passive lysis buffer (Promega, Southampton, UK) was added. The cells were left to lyse for 40 mins on a Titramax 100 shaker at 900 rpm (Heidolph Instruments, Schwabach, Germany) and then firefly and renilla luciferase activity was assayed using the Dual luciferase kit (cat#E1960, Promega) and a Tropix Luminometer (Applied Biosystems, Warrington, UK). Renilla luciferase was expressed from a constitutive promoter and was used to normalise data. Firefire luciferase expression was driven from the GAL4 promoter and relied on reconstitution of transcription factor activity as a result of protein:protein interaction. The signal was calculated as relative light units (RLU) by calculating the ratio of the firefire luciferase signal to the renilla luciferase signal.

TDOA and α-paxillin interacted in mammalian 2-hybrid, again confirming the direct physical association between the two proteins discovered by yeast 2-hybrid. The interaction occurred in both “orientations” of mammalian 2-hybrid, i.e. regardless of whether TDOA fused to the DNA binding domain or activation domain (and vice versa for α-paxillin). However, we observed that when α-paxillin was expressed in the mammalian 2-hybrid system as a fusion with the DNA binding domain of GAL4 (in the pBind construct) that it acted as a transcriptional activator. This was not due to a general effect of α-paxillin over-expression on gene transcription because the α-paxillin/VP16 fusion did not show any additional reporter gene activation compared to controls.

Paxillin is normally associated with integrin signal transduction through focal adhesions (See for example; Panetti. Frontiers in Bioscience 7:143-150, 2002). However, there is evidence that in Xenopus A6 cells paxillin translocates to the nucleus when cells are grown on certain extracellular matrices (Ogawa et al., Biochemical & Biophysical Research Communications. 304: 676-683, 2003). Additionally, paxillin can act to potentiate gene transcription through the androgen receptor (Kasai. et al., Cancer Research. 63: 4927-4935, 2003) and so our observation that α-paxillin acted as a transcriptional activator in mammalian 2-hybrid was probably not an artefact.

Using mammalian 2-hybrid we were also able to demonstrate that TDOA could physically interact with itself and hence may form multimers in the cell. This would be consistent with TDOA having a functional Ti domain

### EXAMPLE 8

We next considered the effect that over-expression of wild-type TDOA would have in the mammalian 2-hybrid system. TDOA was cloned into the mammalian expression vector pCEP4 (Invitrogen) and this introduced into the mammalian 2-hybrid experiment performed with TDOA and α-paxillin. The addition of extra TDOA into this system resulted in an increase in the signal from mammalian 2-hybrid (see FIG. 1). This was not due to TDOA causing an increase in the mammalian 2-hybrid signal on its own and hence indicated that TDOA probably bound to α-paxillin as a multimer.

TDOA cDNA (SEQ ID NO: 3) was cloned into the pAct activation domain plasmid and α-paxillin cDNA (SEQ ID NO: 12) was cloned into the pBind DNA binding domain plasmid. Both vectors were transfected into HEK 293T cells together with the indicated amount of pCEP4 TDOA (a plasmid expressing wild-type TDOA under the control of the CMV promoter).

All of the SNPs associated with this region on Chromosome 11, are found within the non-coding regions of TDOA (introns or promoter/enhancer regions) and we reasoned that these SNPs may affect either the expression level of TDOA or the stability of TDOA mRNA.

### EXAMPLE 9

### mRNA Levels of TDOA in Cartilage from OA Patients and Normal Post-Mortem Samples RNA Isolation

Osteoarthritic cartilage was obtained from consented total knee joint replacement samples or consented knee articular cartilage taken post mortem. Cartilage was snap frozen and ground under liquid nitrogen using a Glen Creston Spex mill. RNA was extracted from the ground cartilage using a standard TRIzol extraction method (Invitrogen) following manufacturer's protocols. The RNA was purified using a Qiagen RNeasy minicolumn (Qiagen), and treated with DNase. RNA was quantified using an Agilent Bioanalyser 2100 with the RNA Nano6000 chip.

### Quantitative PCR

TaqMan real-time quantitative polymerase chain reaction (PCR) assay was performed on an ABI Prism 7700 Sequence Detection System, according to the manufacturer's protocol (Applied Biosystems). The sequences of the TaqMan RT-PCR assay primers used are identified as SEQ ID Nos: 13 and 14; the probe employed had the sequence disclosed in SEQ ID NO: 15.

25 ng of RNA was mixed with relevant forward/reverse primer, fluorescent probes and Taqman Quantitect Probe Master-Mix and RT enzyme mix (Qiagen). Samples were incubated for an initial reverse transcription reaction at 50° C. for 30 minutes and then at 95° C. for 15 minutes, followed by 40 cycles at 95° C. for 15 seconds and 60° C. for 1 minute. Relative quantitation of target RNA was carried out by Taqman using SDS v1.9 software.

### Results

### Expression of TDOA is Upregulated in Osteoarthritic Cartilage

Quantitative RT-PCR performed to establish expression levels of TDOA mRNA in a panel of 9 osteoarthritic cartilage samples (taken from patients undergoing total knee replacement) and normal cartilage taken post mortem, revealed enhanced expression of TDOA in osteoarthritic cartilage (FIG. 2). Thus, TDOA expression levels may be important in contributing to its phenotype in OA.

The inventors next considered the effect that over-expression of TDOA would have on the distribution of α-paxillin in primary human chondrocytes.

### EXAMPLE 10

TDOA adenovirus (Adv-TDOA) and a control virus expressing green fluorescent protein (Adv-GFP) were used for gene delivery. Replication-defective adenovirus (Ad5 c20) encoding full length TDOA and GFP (control) were produced by Galapagos Genomics (2301 CA Leiden, The Netherlands). Primary chondrocytes at passage 2 following isolation were transduced with adenovirus at a multiplicity of infection of 200:1 and 500:1 for 6 hours. Cells were recovered overnight then cultured in serum free DMEM for 24 hours prior to plating on fibronectin coated chamber slides.

Cells were incubated at 37° C. for 1 hour 30′ to allow attachment of the chondrocytes then fixed in 10% neutral buffered formalin for 15′ at room temperature. Washed twice in PBS and permeabilised in 0.1% Triton/PBS for 5′.

Slides were blocked in 3% BSA/PBS overnight at 4° C. Anti-paxillin antibody (BD Transduction Cat# 610052) was added at 1:10,000 in 3% BSA and incubated for 1 hour. Alexa-488 anti-mouse (Molecular probes A-11001) as secondary antibody was applied at 1:500 for 40′ in the dark. Stained cells were mounted in VectaShield antifade (Cat # H-1000) and viewed by fluorescence microscopy at 495 nm.

Cells were harvested for RNA isolation at between 24 h to 72 h post-infection.

TDOA over-expression resulted in increased staining of α-paxillin in the nucleus of primary human chondrocytes. Thus, increased expression of TDOA in OA patients might result in abnormal distribution of α-paxillin in chondrocytes. Based on our observations that α-paxillin has the potential to act as a transcriptional regulator, this in turn might result in changes in downstream genes.

### SUMMARY AND CONCLUSIONS

We have shown that TDOA binds to α-paxillin, a protein involved in integrin signal transduction which itself is a process with strong links to OA (See Peters et al. for a review (Osteoarthritis & Cartilage. 10:831-835 2002)).

We identified the TDOA: α-paxillin interaction using yeast 2-hybrid and then confirmed it by both IP/Western blot and mammalian 2-hybrid. The binding of TDOA to α-paxillin may be as a multimer. We also demonstrated that α-paxillin can act as a transcriptional activator when fused to the DNA binding domain of the GAL4 transcription factor. TDOA mRNA is expressed to a higher-level in the cartilage from OA patients compared to normal post-mortem material. When over-expressed in primary Human chondrocytes TDOA resulted in an abnormal distribution of α-paxillin in the cell, with more of the protein being present in the nucleus.

This data supports the view that one of the functions of TDOA is to regulate α-paxillin and that in OA patients this normal regulation is disrupted due to increased expression of TDOA. This results in an increase in α-paxillin in the nucleus with a subsequent alteration in gene transcription that contributes to the disease state. Disruption of the TDOA: α-paxillin interaction in OA patients may therefore prove to be beneficial in the treatment of OA.

1. A method for the diagnosis of a polymorphism in TDOA, which method comprises determining the sequence of the human at one or more polymorphic position and determining the status of the human by reference to the polymorphism in TDOA.

2. The method according to claim 1, wherein the polymorphism is selected from the group consisting of a polymorphism at position: 30983, 38160, 12523 . . . 12542 and 32945 (each according to SEQ ID NO: 1).

3. A method for assessing the predisposition and/or susceptibility to develop osteoarthritis in a human, which method comprises:
i) determining the sequence of the nucleic acid of the human at one or more of positions: 30983, 38160, 12523 . . . 12542 and 32945 (each according to SEQ ID NO: 1), or a polymorphism in linkage disequilibrium above D′ 0.9 therewith; and,
ii) determining the status of the human by reference to polymorphism(s) present.

4. The method as claimed in claim 1, wherein the presence of an adenine at position 30983 and/or a guanine at position 38160 and or a deletion of the sequence from positions 12523-12542 (each according to the location in SEQ ID NO: 1) is indicative that the human has a predisposition and/or susceptibility to develop OA.

5. A diagnostic kit for diagnosing or prognosing or monitoring OA comprising, one or more diagnostic probe(s) and/or diagnostic primer(s) and/or antibodies capable of selectively hybridising or binding to TDOA.

6. The kit according to claim 5, wherein the primers and probes are capable of detecting a polymorphism at a position selected from the group consisting of: 30983, 38160, 12523 . . . 12542 and 32945 (each according to SEQ ID NO: 1).

7. A method for identifying a compound of potential therapeutic or prophylactic benefit, which method comprises subjecting one or more test compounds to a screen comprising a TDOA polypeptide and determining the ability of the test compound(s) to bind to, block or modulate the polypeptide or inhibit an activity of the polypeptide.

8. The method according to claim 7, wherein the TDOA polypeptide is one that comprises the amino acid sequence shown in SEQ ID NO: 2, or is a homologue thereof or a fragment of either.

9. The method according to claim 7, which utilises a TDOA polypeptide that comprises one or more of the polymorphisms identified in Table 2.

10. The method according to claim 7, wherein the an activity of the polypeptide is selected from the ability to form TDOA multimers and the ability to bind α-paxillin.

11. The method according to claim 7, wherein the potential therapeutic or prophylactic benefit relates to the treatment of OA.

12. A method for identifying potential disease modifying anti OA compounds comprising:
i) contacting an assay system capable of detecting the effect of a test compound against expression level of TDOA with a test compound; and,
ii) assessing the change in expression level of TDOA;
wherein a change in expression level in the presence of the test compound, relative to the absence of the test compound, indicates that the test compound is a compound with therapeutic potential in treating OA.

13. A method of screening for a compound potentially useful in the treatment of OA, which comprises assaying the compound for its ability to directly or indirectly modulate the activity or amount of TDOA.

14. The method according to claim 13, wherein the assay comprises a cell capable of expressing the TDOA polypeptide, or a cell-membrane preparation comprising TDOA polypeptide.

15. The method according to claim 13, wherein the cell is engineered to express the TDOA polypeptide.

16. The method as claimed in claim 13, wherein the activity or amount of TDOA is determined by the method selected from:
(i) measurement of TDOA activity using a cell, cell line or tissue which expresses the TDOA polypeptide or using purified TDOA polypeptide; and
(ii) measurement of TDOA transcription or translation in the cell, cell line or tissue extract expressing the TDOA polypeptide.

17. A method for identifying a compound of potential therapeutic or prophylactic benefit, which method comprises measuring the ability of a test compound to interfere with or inhibit the binding of TDOA to α-paxillin or to itself.

18. The use of a compound able to interfere with or inhibit the binding of TDOA to α-paxillin or TDOA to itself in the preparation of a medicament for the treatment of OA.

19. A method of treating a patient suffering from OA comprising administering to the subject in need of treatment an effective amount of a small molecule drug acting on the TDOA protein or an anti-sense oligonucleotide acting against the TDOA mRNA.

20. The method according to claim 19, wherein the small molecule drug is capable of inhibiting the interaction or binding of TDOA to α-paxillin or to itself.


SEQUENCE LISTING (15 sequences)

SEQ ID NO: 1 - DNA, 49199 bp, Homo sapiens
ccccacctcagcctcctgagtaactgggaccacagggatgtggcaacgtgtgtgtgtgtgtgtgtagagatgggatctccctatattgtccatgctggtttcaaactcctgggctcaagtgatcttcctgcctcagcctcccaaagtattgggattacagttgtgcgccaccatgcccagctacttagatgttttaaaggcactttgcattaattttctatttctgctgtaagaaattaccataaacttagtagcttaaaacaacacaaatttattctcttacagttctgtaaaccagaaatctaaaatcagtcactggagtaaagtcaaggtgtcatttggattggttgcttccagaggctctgcagggagattgtttttcttgtctttttcagcttttagaagttgcctgcattccttggccccttcctcacatcattccaaccaacttccatccttacaatcgattactttcctgtaatagctacagtcaaatgggcagctgaagatgatgatgatgatgatcttgacaccgagaagcagaagaccaatgaagatgaccagacagcaaaaaaggataagttaaaagaaggtaaaagaaggccactcgtacttgctgtctcagtatctaaaggtggtcaccttccagtagagaggcccacccactgcagaccgtgccaccgaaggtgacatgcactttctgccacccatccaaaaccgtaacgtgaatctgcaacaggagaggaaagaagaaccaaaacttccaaggccttgcattttttcttaaaagcatttttttttttttttggagatggagtctcactctattgcctggagtgcagtggcgtaatttcggctcactgaagcctctgcttcccgggtttaagaaattctctgcctcagcctcccaagtagctgggattacaggcacatgccaccacacctgtctaatttttgtatttttaggagagacagggtttcaccgtgttggccaggctggtcttgaactcctgacctcgtgatccacctgccttggcctcccaaagtgctgggattacaggcctgagccactgcacctagccaaaagcattttttaaagaaaaattggtctatattttttgtttctactttatatctttgtacatatttttagagtgcaggcatttttaataatcttggatgaccaaagcagctttcagaatgttctttgcccaacttctgactttacttgtagtgtgaccatgttcattagaatctcaaaggaggaagaaaagaaaaaacaaacttgtaaaaaatgaaaaatagatctggcgaggtgattcacggctgtaagcccagcactttgagaggctaaggcgggtggatcacctgaggtcaggagttcgagaccagcctggccaacatggtgaaatcctatctctactaaatatacaaaaattagccaggtgtggtggtgggtgcctgtagtcccagctactcaggaggctgaggcaggagaatctggaaccccggaggcagaggttgcagtgagccgagatcgcaccattgcactccagcctgggcgacagagtgagactatctgaaaaaaagcaaaaataggccaggcatggtggctcatgccttgatcccagcactttggtaagcctaggcaggtggatcacttgaggtcaggggttcgagaccagcttagccaacatggtgaaaacctgtctctactgaaaatacaaaaattagccaggcgtggtggtgggtacctgtaatcccagctacgcgggaggctgaggcatgagaattgcttgaactcaggaggcggaggttgcagtgagcagcagtcatgccattgcactccagcctggtgacagagtaagactctgtctcaaaaataaataaatgcaaaaacaacaaatcttattttttcagcctgttcgatgtatgtgtgaaacaatgttgtccaacaataaacgggaatcttatttttctgaggttttttcccctaacttcatttctagtgaattttctgagttctaacaaaaaatggtagagacagttgtcatgaattgtaccccttttctcattctttctgctttaggcttgaacatgaggcataaagctagactcagctgttaggaaaaactgcctcatgctggccctgacatcattgagctaataaacaaatccctctacctccagacttattatgtgaggaaacacacacacacacacacacacacacacacacacacacacacgtgttactgaaacaccaggggttccgatctagttcctgttccttgcttcacaaaaagccaatcactgagatgattattgccaaagaataaggctttaatcaggtgctgcagagaaacagatgggagatcaatctcaaacttgggtccctgactgactaaaactaggtttatatagcaggaaagaaatgtaataatgtgttaaaaaaaaaaaaaaaaggaactagggaggaataaggaagcaatcatgatgaatgaggggcttggcatctcattttctggatgctgtgatccggtgagtttcagttctttgatactttttttgagagacctgaaggtcatttcctgaggaaggaactcagataaatataactttccagctttaagaccagaagggtcaatttctatgtttgtccaaaaaaaaactaaggtactattgggtcaggttcacacacgcccacaccacccttaggtgtttaagccacagttggtcagtttacctgttcttccagcaaaagcatttctcactgctcttgccaggtcctgtctatttaacctcctagatgtctctcacattcatgcattcctcttcacccacctttgccactctaggcataatgacctgctttcagcctttggtacatgccatgttccctcctgtcactgccctttatacacgcccttccctctgtctggaatgttcttccttctccccttaggctagttagctcttcatcctttggcttttatagcaagcactgcttccccaagacagccttccccaacctccccttatcctaccctgggcacctgtcagctggatgcaattttatactatgtgtgtgattatccaattatctcactagttattcactccatgaggacagggaccttttctgttttattcatcattgtagcagcagcaacccacatagcagctgacacaacagatacgctctatatttgttgaatggcatgaatgtctaaaataggattccaacccagtctctcctacctgagtctctgcttatcctcagtagctgggactacaggcatggcatgtgccaccatgcctcttcggttcacctttaaaaaataaaagaattggccacatacaatggctcacacctgtaatcccagcacttcgggaggccaaggcaggtggatcacaaggttaggagttcgagaccagcttggccaacatggcaaaaaccccgtctctactaaaaatacaaaaattagccagacgtggcaggcacctgtaatcccagctacttgggaggctgaggcgggagaattgcttgaaaccggaaggcagaggtttcagtgagccaagatcttgccactggactccagcctaggtgaaagagcaaaactctgtctcaaaaaaaaaaaagaagaatgaaaacaagtctctaggcaatcagaacccatagagcggctgcagtttgtgagatcatcaaaatctggacactccctttcaaagaaacgtgtagtggtcaggaaagctttccagtcaccggcaatggccaggaactggagtagagtggactgatttcatagaaagaaaagaggccaggtcacatggtttatgaaacacttcaacgatctgctcagagttttcagcttgaagacccctatgaaactgaccttggcccttttctgcaatgagtatatttgagacatggggagaaattgcagaaaggtgccgatgtcagttttatagggtcttcaagattatgattaattgaataagttatatgtttttagacttcgggatgattgtttataatgtcacttggagggtaatgaaactcagaaagaaacaggccaggtgtggtggctcatccctgtaatcccagcattttcggaggccgaggtgggaggatcgcttgagctcaggagttccacaccagtctggccaacatagcaaaaacttgtctctactaaaaataaaaataaaataaaaaaattagccaggcatgatgatgcatacctgtagttccagctactggggaggctgaggtgggaggattgtttaagcttgggagattgaggctgcagtgagctgtgattgtgcgccactgcattccagccttggcaagagagtgagaccctgtctcaaaaaaagaaagaaagaaacagtccagttccaagtttaaaattagaagtgaatagtattgcatttatatgccatcctagctatttattgctgcataacaagttacccaaaacttagcagcctagaaacatttattatttcagagtttctgtggtcaggaattcatgagtggcttagctaggtggttctggttcaggttgcagccaagatgacaccaggggctgtagtcaactgaaggctccactggagctgtagtatccacttccaaggccaacagaagccctcagttccttatcacatgatccaccttccttggcctcccaaagtgctgggattactggcatgagccacagccccaggccccaaatgcccttttcctttattgtatgtcaggctcctcctatcctatgacaaatcctgcatgacccagttcagatgatctatgaaattttcccaaagcacttcatgcaggtttcttttcagaatttacaattgtaaccccgatgacagctaagccctccaagattcttcaaatctaaataaaactgactttgcccaaccatttagtctcaatttgacagcctgtggaagccaaggctttcccttgtatttctgcagagtctcagaggcatacattttcacatcacctcttttctcctggacttcctaattctcagttgtggcctgtgttcttaccttataacccagattggagattcagctccttcactgctttccaggcaaaacctgctgatacacagtatttatgagtacagagactttactgacgagcacagcacacacagcaaatcctttaaatctcaaggcatcaggctggccctccctggtcctctttgactctttggccatgtggtgtccccctgttgccctttcctggtgcagtttcagtctccccatttttctgatcttccctcactctctgaaacaacaaacctcttcgaaagcctttcttgtgtagagtagttccagggcgctttctgttcttggcctctccttctacccaagaccttatgaaggtgttattccaaagaagacaattcctttcccaagaggagttcttggaccaacaggataataattttccagtcatcttgtatgtttataagcatgtcttagttcaggctgctataccgaagtaccacagactgagtgtcttataaacaacagacaaacagacatttattcctcacagttctggaggctggaagtctgagatccaggtccaggaagcgggtctggtgagggctgtcttccaggttgcagactgccaacttcttgtatcctcacatggtggaaagagagtgagagcgctttctgggctctctttcatttttatttacttatttatttatttttgagacagagactcactgtgtcacccaggctagaatgcagtggcatgatctctgctcactgcaacctctgccaccagggttcaagtgattctcctgctttaacctcccaagtagctgggattacaggtgccagccaccatgcctgggtcatttttgtatttttagtagagatggagtttcactgtgttggtcaggctggtcttgaacccctgacctcaagcaatccacctgcttcagcctcccaaagtgctgagattacaggtgtgagccaccgctcccagccagtgggctgtcttttataagggcactaattctactcatgagggctcaaccctcataacctaattactttagacaggcccccacctccctataccatcacattgaaggttaagatttcaacatatcaactggggtagggggctcaaacattcagtctataacagagtgtgcctcaccccatgggtctgaatcaggtctggtctgagttggttgtcccagatccctgcacagagcttggaatatagtaggccttcagtaacatttgttgaataaatgaatttgtgataaatgagaatgattgattaaaaaatactgaataaaataatgaatcagctaggcgtggtggctcacgcctataatcccagcactttgggaggccgaggcgggcggatcacctgaggtcaggagtttgagactagtctggccaacatggtaaaaccccaactctactaaaaatacaaaaaattagccaggcatggtggtgggtgcctgtaatctcagctacttggcaggctgaggcaggagaatcacttgaacccgggaagcagaggttgtagtgagctgagatcgcgccattgcactccagcctgggcaacaagagcgaaatttcgtgtcaaaaaaaaaaaaaaaagaatgaatcagttgacattaattgacctgatggttcccagggatctttgccctgcaaaagaagaagctgacagaagttctagtcctaattctagtcccagttttgcctctcagttgcgtgtaatcatgggcaggtcacttttcctctctgtacctcaggtgaaaaatgagaaggaggcttcttgttattcctttttgttctgccaaccacacctcattctaatcgtcacctgcttgccacctgatgcttcatgctgggacctggctggcctgctgggtagcttattattaaaatccacctgcctggttaaaaagtgctttcaaagcagagggcccacctggaattgttctagctcatgattcttgttcagccacaccattgcattccttgccttcctttaatgcctgttacaactgcacattgaataagctctttcattctcctcccaatttacgaatgaaatcccatcaatcttttcccatgcccagccctcacgcaggagtgtgtgtctatgtgtttgtgtgtgtgaatgcgtgtacctgcattcacttgcatgcatacacacacaccctcttccttaaccaggtttattgtgtctgaaattttccgaaactttgtataggctttctgaaggaagtagaaaccccatgtagtatttgtgttttttttaagacagggtctcactctgttgcccaggctggagtgcagtggcgtgatcttggctccccgtgtagtattcgtagcaaggctttggtcatagttgctttttcactgtttgaggccttcactggagctatctaccatgagtcctcagaggccaggatttccaaggagtaactgatcagttttcattcgttcttagctcattgatatcactaagcctctgtgtcaataactaatgtaagtatgtatgtatgtatgtaagtgcagtggtgtgatcatagctcactgcagcctcaaactcctgttctcaggtgatcctcccatctcagtctcctgagtagctgggaccacagttacgtgccaccacacctggctaattaaaaaaattttttttgtagagatgggtcttgccatgttgcccaggctggtcttgaactgccaacctcaagcaatcttccctccttggcctcccaaagtgctgggattacaagtgtgagccactatgcctagccaacatctaagatcttttttttttttttgagacagggtctcgctctgtatcccaggctggagttcagtggcacgatctccgctcgctgcaacctccgcctcctgggttcaagtgattctcctgcctcagcctcccaagtagctggattacaggtgcgtaccaccatgcctggctaattttttgtatttttagtagagatggggtttcaccattttggccaggctggtcttaaactcctgatctcaagtgatctgcccacctcggcctccaaaagttcttggattataggcgtgagccaccgcacctggcacaacaactaagatctttcttcccgcctccctgctcccccagagacagtctcgctctgtcaaccaggctggagtgcagtggcgcaatcttggctcactgcaacctccactttccaggttcaagtgatcctcccgtctcagcctcccaagtagctgggattacaggcgcgtgccaccatgcccagctaatttttgtatttttttagagacagggtttcaccatgttggctaggctggaaaactaagatcttaatttgagtgccaaagggtgtcccacagctcctttcctatattttgttattggggcggggccaggacacacatttcagtaatctttgcttttaagggttatgaggtaacttaaaatatggaaaaaaaaaaaacaggtataacacacagaaccttagatttcattgttgccaagctccatgtgaactaggatatgaatagtagcaatgattcaattattattaatagcatacacttactagataccaaacactgggtgaagtgtgatacataagtcatctcatttatgtaaacattgtgaggaaatagagcactggagagttaaggaactcactcgggtactcactgctggtaagtgatgcagccaggattcaacccagcccatcagatggcagcatctgcatctataaccattacactatttctttcttcaagctcccaatttgtgttctgaggagagactaaataaggaaaagaaaaaaatgtcttcccctatttagccaccagacctttgcccatgacttggaagtgtttgctgtggagtctcctatcttgtcttttttttttttttttgagatggagtctcgctctgtctcccaggctggagtgcagtggcatgatctcggctcactgcaatctcctccttctgggcccaagcgattctcctgcctcattcttcccagtagctgggattataggcttccgccccaacgcctggctaatttttgtatttttagtagagacaaggtttctctattggtcaggctggtctggaactcctgacctcaagtgatcctccccctttggtatcccaaagtgctgggattacaggcgtgagccactgcacctagcctcctatcttgtcttcatgagtgttcagaattttgtggtgcactgatcagaagcccagaggaatctagacttcattgaggtgcaccttggtatttgaatcccactctctctttttgtttttattttttggtggagtgggagggtaggggcagacagagtctcactctgttgcccaggccaaagtgcagtgggcacagctcactgcagccttgacctcacaggcttaaatcatcctcccacctcagcctccggagtagctaggactataggtgcaccaccacacctggctaatttttgtattttttgtagaactgaggtttcgccatgttgcccaggctggtctcaaactcctgggctcaagcaatctgcccgcctcggcctcccaaagtgctgagattacagtcatgagctactgtgcccagcccaaattcctctttatcagcatcagatcatatcaatatcaattttataaagaccattaatatcaaccaggagaaggatgaaagcaaaatgttctaagagcaaagggactgctgattatcatcaagtgaccctatttcacaggaagaaaatgtggatgacattttatgaccaacagcattgtttatttaaccttggagcacaatagagaaaaacttttgactccaacccccagtccctgaaggcagaagtctgaaataccctctcaattctgtttatggccttaaagcatttgtcttagcctatattctccccaaagcagggcctgagacaaaggcttatgtgtgagtagtttaatttgctgtgctgatctcaggaagcaggagctatgcttgcagaaaatgaaatagggagtgcaaatataaggataagatattaactactactgaggacaactgattactcaagcatgggagaccttctgagagaccatataggatgcatttaaggactgtcttgctcaggggaagagagggagaaaaggttatccatcagtttccctctcccattggtcaagactggccctagggaacatttatgtttcctgtaaatgagtgctaagtggactcccacagacatcaggcatggcagctcaaagagactggaaggcagaaaatgaaagacatgagacgggcctgaggccaggcattttcaggttacatcttcatgaagatggtcactgctgcacaatggctggaataagaggtgagactgacaagatatgaagtgggacatgagaggtgtttgacagagtccaacccttgcaccattcaagcccacttaaaatgcccttgtgtaaatttaatttgtcatacggtcgtcttcagtatggcagttggctgcgttctctgcaaacacttgataatccaagcaaattagtgaaacaagttataacacttgctgctacagttgatcccaaagctgtaattgatattcattatctgcttcttctaccactcaatctagatttctcttattctcatctagcactttggctggcctggatttcttgcttggtggtgggactatgactgtccctgaagtccttatgattgccttacccttgatatgatcatgattactacaaatagcctgttcatcattaccactggatttgttcctgagctgacacttaactgagccttaccagaacattccaccatcatctgccagggtctgttttattgcagaagccatagaggtgatctgaatagagatataatgggcataaacaggcttgtgtcctctaagtctgttctgatggtgttaatctctgcagttctttctgggatgcaatattgcttctactgatgggggcagtgaagtgtctgggtaacttcaggggcttccacttggccctttctactacagactcttactcggctggatgtggtggctcacgcctgtaattccagcactttgggaggccgaggcgggcggatcacttgaggtcaggagttcgagatcagcctggccaatgtggtgaaaccccatctctactaacattagagactacaaaaattagccgtgtgtggtggtggcatgcctgtaattccagctactcaggaggctgaggcaggggaatcgcttgaacccaggaggtggaggttgcagtgagccgggatcaagccattgtactccagcctgggtgacagaacaagactctgtctcaaaaaaaaaaaagattcttactcctcaggtcagaaaaacaacacaagaattctaatatctgctaaatatatatctgctgatctctacttccagaaactggggaattaaccacagggtgggtccgtgaatgcatagaaccaatgtgaaatgcatttgggctagaagtcattttatcacttggctttcatcattcaaattcatcatctaaaaatggggggctgcatgcggtgcctcacacctgtaattccagcactttgggaggctgaggtgggtggatcatctgaggtgaggagttcaagactcatcctggcccacaatgcaaaacccgtctgcactaaaaatacaaacattagccaggcgtggtggcgggcacttgtagtcccagctactcgagaggctgaggcgggagaatcacttgaacccaggagatggaggttgcagtcagccgagattgtgccacttcactccagcctgggtgacggagcgagactccctgtcaataaataaataaataaataaataatgggggtgacagttcttatcacttacgggttttgtgaggattaaatggtatgtgtaaacaccaaaacagctgttgacacacagtaggtgttcaagaaatagtactgtgataggacatttttctacagaaatcctgactcccgctttggatgatttccttgaatcctgagttttttgaattccccaaattgtcctccatggtactacatagtcattaagaacaaagatttaggagccagatcattggggttttgaatcttggctctaccagtcactagctgttggatgttgggcaagtggtttaatctatctgtgattcaatttctttaatctataaaatgagaaaaattatatatctcacagtattgttttggtaattgagttattatatggaaaacacctagaattgcctggcacatagtaagcagaatgtaactgttagctattattattttattgttattttttgagatggggtctcactctgttgcccaggctggagtgcaatggtgagatcatggcttgctgcagcctcaacctcccacgcttaagtgatcctcccacctcagcctcccaagtagctgggactacaggtgcatgccaccacacccagctaatttttgtatatttgtagagacagggtttcaccatgttacccaggctggtcttgagtcctgggctcaaacaatccgcacactttggcctcccaaagtgctgggattacaggtgtgagccaccatgcctggcctattattattattaattggccaacactaccacctagaatttcttactagctggctttttttcccccatccgttgcttagctacatatatctcccctctcactcttctctttctccttccaaatcaaaaggttacagcctgctggtggccattctgacaggctgggaagcataggctccagttagaagccagaaacaggctcttcctctccttccaaatcatacctgggggactcagcttttctgggaaacgtaataatagaaacatttacccagcacttactcagagtattgtgctaaattctttccatggatgaactcttaattttcacaacaatcttaagaggtagatataaatattatgcccactttatagatagggaaaattacagtttcagagagacatgacaacttacctaaggtcacattctagaaattggtagagttgggatttgagcccaggtttgtgatattccctagcctattctaaccataggaatatggcattgcgtagtagtaaatttatgcactctagcatcagactgtgttcacatgctggactcacagctgtgacttactactgccttgcctacttaagtggcttctgaaaatatttggcctttccattggagatggagacaaatgtgggcagtaatgtttttaaatagagttttccccaatacccaacatagaatctgccgcagagcagatgcctcataaacattggctcagaaaataaatatattcacgacactattgtaggcacggtgtgggttacaaaggcagttgattgattacttatttattgcaaggaattggctgttttgctcgtcagggctggcttggcaagttaaaaatctgcagggcaaaccagcaggctggaaactcaggcaggagtggaagcagcaatccacagtaagtttcttccccaaggaagacttcagtttggtctttaggcctttacactgattggatgaggcacacccagattattgaagacagtctcttttattcaaaagtcaactgactgtacatgttaaccacatctacaaaataccatcacagcaacacctagattcgtgtttgattaataaagtaggtactatatcctagccatattgacacataaaactaaacttcatggtcagacttggtggctcacgcctgtaatcccagcattttgggaggccaagacagggggattgcttgagtccaggagttggagaccagcctgagcaacatggcaaaaccctgtcactacaaaaaatttaaaaaactagccaggtgaggtggcgggagcctgtaatcccagctacccaggaagcttgagccctggcagtcgaggttgcagtgagccaagatcgcgtcactgcactccagcctgagtaacagagtgagaccctgtctcaaaaacagacaatcaaaaaactaaactaaactaaccttcacttaattttttcaagttcgtttcatcagctataaaattgagatactgatccttacctccttacctcctatatgaaaatgcacattaggtagcagaactttgcatgaagcagagcaacagggcttaacaggtcttagcgctcagtctacaagaatcagtggggaggccgttgcaatgctttgacgagcagggtttggtttgaataagaatatatttttccaacatggatattttgtaatataagcacatgggtggcggggaaacagttcaaactggcaatcaatgaagaggttgtctcactctaggcctttccctcagaagtaatcgttgctgatagtttctgactatttttccagaattttgttctatatgcatgcacgctcaatgcctaatcacactcagccttttgtccccttgcaccttgctttttaaatttaatggtatgtcttggagaatgttccatttcagcatgtacaaatctacttcttttaaaaaatggctgcaaagggctgggcgtggtggctcacgtctgtaatcctggcactttgggaggccaaggcaggcggattgcctgagctcaggagtttgagaccagtctgggcaacacagtgaaaccccatcaatacaaaaaaaaaaaaaaattagccgggtgtggcagcgtgtgcctgtagtcccacctactcgggaggctgaggcaggagaattgcttgaacccaggaggcggaggttgtagtgagctgagatcacaccactgcactccagcctgggcgacagagcgagactctgtgtcttaaaaaaaattggctgcaaagtattccattgtgttgttaaatactaatttacttatttaataagttctcaactgatggtaatttaggttaccaacttttttgctattataaataatacaagggtaaacatctttgtacatacatacatgtttgcataatgtgtaagaagagatgtaagataaatttcgagaactggaaatactgcattaaagggtttgcacatttaaaatgtcaatgtatagtgccactttatctttcaaagagattgcattagtttatacccaaccaatagtatacgagagtgctgtttccccatgcccttatcaacactgccaccttggatttaaacctacttgttttcctttaaaataccaaaatgggcatttgaaaaaaagaggcatgatttgttccttggcaagagctatattttgtttgagtttgatctttctgatagcagacctaggccttgtgccctgtcctgtggtcccatctctgaggagctgggcagatgacctgaccacaggactcaatcaggatagcttcaggtcaccaggccaaagggtaggccagacccgggcctgtgcctagcctgatgatggaaatgcaaacaagccaagttctataatagaacacagagccattgccgggaaaccacaaattgctgacttgctcctgcaaacaatcaatattcagaagctgagtccaggaattctaacgatgcatcctgcacaaagggctgagtgggggaggatgtgcttttagaaaaaaacccagcaccagaagccctggtgctgcttcttagagggccatcaagaaggcccttggtcccatcaccacgccctctgacaatcccagaaccctaggctctcggagccagagtagatctgggaaattatcctgccctgagagagtccaggcaaagaatcaagggagttccaaggagcaagtgctcacttcggtgggatagtgcagggcagtggttagtggctacacttgggctcaaatcctgactcagtcaccagctttctgaccttggacacattgcttcacatctctcagcctcagttttctcatctatagaatgaagacaataacacctatctcagaaggtcattatgaagattcaactgaataattatgtttgtaaataatttagcatggcaactggctttttgtaaggtcgtaataaattattattaatattcattttttttttgagacagagtctccctctgtcacccaggttggagtgcagtggcactatctaggcttgaatcctcctgggttcaagtgattcttcggtctcagcctcccaagtagctgggactacaggcatacactagcacgcccagcaaattttttgtatttttagtagagatgtggtttcgccatgctggccaggctggtctcaaactcctgagctcaagtgatccactgaccttggcctctcaaagtgctaagattacaggtgtgagccaccatgcctggcctatattgttatgaataaacaataggatatcaggagtggggactaaggagaggccatttgcactagaccttgaagaatttggaagatttggtaattagaatgattaaagggatgaagttcctgtgcaaaggttctcttctcgtgtctctcctggcctgaccaggaggcatgctggggtctcagcagtagaccactgcagaggagggtgggtatatgtaggggataattccactgcagaacatgtcaaatagtccttgcttaaatggtatctacaatgacatttgcatctgtgttccaaattcatccccatatcctcctgtctcagcctcccaaagttctgagattacaggcatgagccactgtgcccagcctccatccacatttgaatgcaggctccatcagggcaggcccctttactgttaaaattctgcgtgatgatgaagggcacatgcctgcacggatttcctctactctgttccaccttagctgcttccctccctcagcaacgcctggcatccctactctggggtcgttacttagaggcaggatatggctccaatttagcaccattatccccaggaccagatagacagtaattggaatgaatcaatataacatacaaaactcttcataatctgggttcagtatatttttttagccattctcttagcctagactccagctagtctctagcatagactacatagaataatggtaacattgggctgggggtggtggctcatgcctgtaatctcagcactttgggaggctgaggtgggtggatcacttgaggccaggagtccaagacaagcctggccaacatggtgaaaccccgtctctactaaaaatacaaaaagtagccacgcatggtggtgcacgcctgtagtcccagctacttgggaggctggggcaggagaagagcttgaacccgggaggcggaggttgcagtgagccaagatcgtaccactgcactccagcctgggcaacagagtgagactctctctcacaaaacaaaaacaaaacaaaacaaaacaaaagaataatggtaatattgatcatcattcactgagtgtgtaccagtcattgttctagcactttacatgcattaattcaccgaatccttacagaaccctatcagttaagtattattattatctccattggtctggtgagaaaattagacccagaaaggtgaaacaaattcctgatagagcaaggacttgaagccaggtggtctgcctggaccctgaaccattcccagccactcctctctgttgcctctgctgcagagtggctttgctctttcaagtcgctgagcctctgctctgctgcaatgcccttccccaacttctggctctagcaaactctacttgccctttgaagcccagcccagatactgcatccctggccccacctgcttctccaaccaggggaaggaggttgtccctgtctccactttgttccccttacaagttgctcccagtcctggatggggaggtcgtctgaaacaggaccctgactcacgcctcagtatccccagccctcagcacagtgcttgtctgtgggtctttcatttgtgttttcaacaaacatgaacgagttgaatgagtaactccactgcttcacacacaaggaaacaaatccagagaggcagagggacctgtccatatcacacagccagtgagagaaagagctgaggccagactccagggctcctgacacccagccctgctctctctcacgccatcccaactgtgtgtggaatgtttggaaaagccttcatgcatctttgagcctttcttaattctgtcttacatttatttcatcccaaagaggacttctttgagaatgctggtgggagagtgaattttggctcgtcttgtctgaagtcatgaggcaacagggaaaaacagacaaacaatcctgagatgtgttttactgacccggtcctcaaaacagcagaacaaaacactccacactcaagctggggctgctagccagcagggcactgagtgaagctggacagaattcactagggatcctcctcccttttgcttaaaatggtggggactttggagaggaagtgttttctggttagtctgaattttctttttttttttcctttttttttgagatggagtcccactctgctccccaggctagagtgcagtggcacaatctcagctcactgcaacctccgcctcctgggttcaagcgattctcctgcctcagcctcccgagtagctgggattacaggcgtgcaccaccacacctggctaattttcatttttttagtagagatggggttttaccatgttggccaggctggtcttgaactcctgacctcaggtgatccatccgcctcagcctttcaaagtgctgggattacaggcttgagccactgtgcctggccctttttttttctttttctttctttttgacagggtcttgctctatcacccaggctggagtgcagtggcacaatcatggctcattcccacctcagcctcccaggcccaaatgatcctcccacctcacccgtgtagctgggaccacagctgtgcaccaccatgcctggataatttttaaattttttgtagagatgaggtctccctatgtcacccaggctgatctcaaactcctaagctcaagggatcctcctgcctctacctcccaaaatgctgggattacaggcttgagccactgtgcctggcccttttttttctttttctttctttttgacagggtcttgctctaccacccaggctggagtgcagtggcacaatcatggctcattcccacctcagcctcctgggcccaaatgatcctcccacctcacccgtgtagctgggaccacaggcgtgcaccaccatgcctggataatttttaaattttttgtggagatgaggtctccctatgtcacccaggctgatctcaaactcctaagctcaagggatcctcctgcctctacctcccaaaatgctgggattacaggcgtgagccaccacacctggccagtgatgattacttattcatgatgtcctttatggctggaatggccttcctcaccttgtctgcctaggaaacttttacttgtccatcataagggtgttctccttggcaaggcatctctgaccatcccgtttcccctcagtgtggtccctggttctctcagctctctcaccccttttacctttgcccctctaacacaatctgatttatgttgaaattgtttacttgcccatcttccctagtaggtgatgccagctaagaggataggctcaggtctgtgccctcagggcctaacagtgcctgccccatggagactcaggaactggcttctacatccctagatcaggagcactaggaggttgggagaagagaagctttgcagagctgggggctgcacagctcctgggagcacaaatcatatcatatgctattactttgtttgaatggagtccctggagctgtgcagcttggtagctctgggcctctgcctgagacagacttggtcttgaatcttagctctgccacattctggcttcactccttggaccatttaatctgtttaggcctcagtgtcttcacttgtcaatgactgttatgattgtgacctctcagattgttgtcaagattaaatgagatgatacacacaacgatcccgatggtgcctggccagtggtagttgtaacccggtaccggatttttaatgacaagattcataccagggctgagcatcagactatggggttgccttggtagtgtaagaggcagaaaggtaatgtgaagaggcagaaagaacagtgctctaggagacaagaaccctggatacaagggctgtgtgtgtgcacatgtgtgtgtgtactttctctgccctaccattgtggtccatcttcccctctgtcccaggcccatcctggttttgtactccatactgagctggaaaaatttgaggtagaggagcagctaggaacccatctagggtggccaggcctacccgtgcgctgggaagcaggagtgcccttgagcagtggcactgctttgggctggagccatgaggcctcctctgtggtgggggtcatggtttggagcatgggctatggagccaacagcctgggtgcaaaatccctcttagccccttcctcactgttctacctgtctgtgcctcagcttcctcactggattagttcactagggctgctgtaacaaagtgccacagcctgggtggctcaaacagcagaagtcatttcacatagttttggaggctaggagtgtgagatcaaggtgttggcccgattggtgtctagtgagggcctctctcctcggcttgcgggtggctggctttgtggttctatgttcgctattggtgaaataagggacagtgagctcagaaggaagtgaacttccagggtctaaatagcccaggtggaacagtgcacagacctgcagtgacactgcccagctggccaatactggactgtgtttcatcctagacaatttcaagacacaagtttgttttatgttctattaataccgtataagaataaacactagaaattgtaattgtaagacaccatcaattaagtctcactctgatctcagagatgctgaaatttgaaaaaaatatgtgttgtgtcttagaattgatgaaagacagtaggtcctgttttttagcttttggcagtgaggatccactgaggtggagggggggcaggccatgtgtctggggccgcagacgctggctgctgtccagtatcagcgtccacacaggaagtggaatggtgaagcaccatcccagtcaagtctcctcacttggaaaactggctaaatccacggctgggaaacagatctcaaaagctgcacaggtcccacttgagctttttggtgggccttgtaaaatgagccctttgcgtgggcatctgacagacctgggttcttgctctaaagctgctactcatcaaccaagaaattgaaagatgaaaggctccaaagtgcaaagaggggccaggcatggtggctcatgcctgtaatccctccgaggtgtgtggatcactggagcccaggagttcgagaccagcctgggcaatatggtgaaacctgatctctacaaaaaaaaaaaaaaaaacaaaaacaaaaagaagaagaagaaaattagccaggtgtggtggcacacaccagtagccccagctactctggaggctgaggtgggaggatcacttgagcccaggaggtcaaggcttcagtgagccatgatcatgatgccactgcactccagcctgggtgacagaatgagaccctgtctcaataagtaaacaaaagtgcaaagagggctttgccaaatgttgcatgcacaagatggtgtgtgtgtgagagacagggctgggagtcaggggtcccggggttcggttttggcttttccctccctggctgtggacttggccaggtccctctgcctctctggacctgtttcctcgtgggtaaagcagttgagttcagtcactcatccaacaaatgtgaactgggggccctttctgggtggtgtgctgagggctgggggtatacagggtgagatgtggtctccgctcccggagagctccgtccagtgtggagagcaaggagtgaagagagcagagaggaggcagccaggcattctccatgcactgtctcctttaatcctcatcatcaccttatgtgatgggtaacacaggcatttttattgccatttcagagatgcagtcatggagactgagaaagatggtgctgcttaccaaggcagggccagggtttggacctgttctgccttacctaaagccagttctctttcctcttcaccttggatggtcagtcagaagtttccttctagcacagcattctcagattctatgccaaaaaatagcctgtgagtgtcacattctgacaggcttctacaagatccccctcacttagcaaccaagtaagggagagcctctctctgctcaggaatggtggggccgggggtaggggtgcagggtagctcagcttgtctaacctggcttgtcttctaaggaggtggtaagcaccatgtttcaggggagtgagcctccccctcacaaaagctttccatctacactcatgggcatgtcagtggtgtgacatgccatctgagtgattccactttgcctgttcattttatgcccagcagctcttaatacagtccagcagttcatactggaaaatacagaaatcaagggactgctctgtcagtgagcattatgttcacaggttcgtgtcacaggatgaaggggcaggagaagatgctgggggccagtccttagtctgaagcctccatggcagccccatcaagggattttttggtctctgtttgcatatctccctggataggaggatcatgcttcctttcagctctggctagaagagacttctagctggtagagcccaacaataccttgcacattttgccgaggaagctctcaggctgacagtgaagtgatttgctcagggttgcctggtgcgacagtggaaagcctgggtcagagtcttggcactgggaagcctggttctgtccttgacctcaaagttgagtggaaataatcccaagggaggatttaggctggattcttgcctcaaccaggtcatttaacttgaattaaatgatatttaattcaatttaatattagccaaggcctactatgtgccaggcactatacttacacctaatgtgatcacctccctgaaccctagtttcccatctatgaaaaaaaatacctcctgtgcatcttttctcagagccctgttatgaggatgaaatgaagaaaaggaagtaaagtgctttgtaaactgtaaagtgctgtgtccattgaggcattgttgctaaagctggccatcctatctctgcggtccactctgcctggagcagacttcccacttagcactggctaccatatttgaatttgtttatctgctcctcacccccaccagagttcccaggggaggaatgtttgggggcttgtatttgatttcccagaacctcgccctgggcctggcacatggcagatgcttaacagtatttataaatgtcatagtccattcaggctgctataacaaaatgccataaactgggtagcagaaatgtatttcccacagttctggaggctgggaagtccaagtccaagatcagggacaggcagatttggtgtctggtgaagcctggtttcctgcctcatacatagctgtctgtcttccctgtgtcctcatggggtagaaagggcaagggaaccctttggggtcccttttttttgagacggagtttcactcttattgcccaggctagagtgcaatggtgtgatcttagctcaccccaacctctgcctcccgggtttgagcgattcttccgcctcatcctcctgagtagctggcattacaggcatgcgccaccacgtgtagctaattttgtatttttagtagagatggtggtttctccatgttggtcaggctggtctcgaactcccgacctcaggtgacccgcccaacctggcttcccaaagtgctgggattacaggcgtgagccaccatgcccgtttttttttttttttctttttgagacggattcttactctgttgcccaggctggagtgcaatggcacgatcttggctcactgcaacctccacctcccggattcaagcgattctcctgcctcagccttccgagtagctgggattacaggcatgcaccaccatgctcagctaattttcttttcttttttttttttgtatttagtagagatggggtttcaccatgttaggctggtcttgaactcctgacctcaggtgatccacctgcctcggcctcccaaagtgctgggattacagatgttcaccaccgtgtccagccctgcggtcccttttgtaggaacaaatcccattcatgagggttccactctcacgacctaactacctctcaaagcctatccctccaaatatcacactggggttaggtttcaacttaggggtattatagggacacaagcattcagtctgtaaacagaacatgtgatgagctcttgtttctcagacccaaggagatgccaacctgtgggcactggaagacccctggggggagggctagggagagggcaggtgggaagggtgaccactgtggtgaagtagctacattgtatagggtatataccctggggtgcgtctccttgcgccaggaaaatttaggacatgggcacacacgaggagtttaggagcggagttttaataggcagaagagaaagagaaacagctttttctatagagagagaggggtctctgagtggaaaagactggctggtggtggatgcaccagactttatagtccagcttgaggaggtggtgtctgatttatgtagggatcacagattgttttgatcaggtgtgatatttacatagcgcaaggaaggcttgttgccccaccctaatcctatgatgcaaatgaactctccttggctggtgccatcttgtctgctccttactgtacacgtggctggcaaagaagggatgatggagcccccatcttgaacatgtctagtccctccctagtccctagttcctgctgacattcagccatgcaagctcccagcttgtttgtctgtatctgcagcttgactttataggttgctgtttggtagaaaatgattttgggctgcttttcattaaaaagaaaagccttaccgaggactcccatatccttactatctgcctaagtgatttcttcttaactcctattatcagtggggtactctaagcagcgggagtggctttgttatcccagcttggacctgtagtttttggctctcatgtaaatagaaatgaaccctggctgggtgcggtggctcacgcctgtaatatcagcactttgggaggccgaggcgggcagatcatgaggtcaagagatcgagaccatcctggccaacatggtgaaaccccgtctctactaaaaatgcaaacattagctgggcatggtggcacgtgactgtagtcccagctactctggaggctgaggcaggagaatcgcctgaacccgggaggtggaggttgcagtgaattgagatcatgccagtgcactccagctgggcgacagagtaagactccgtcccccacccccctcacaaaaaaagaaaagaaagaaattaacaccaggccaaacagaacttttactcaggcaaggtttcataggcttgcgtctggagcaactgcgcgggagcattgtatgggaaagggatcctggtgctagctcctggaaggacttggctcttgtcattttaaggaagctgaggcgagaaaaaggagtgacatgtaggcatgtagggatggggaacttttagcacctgcgcagtttgactatgtgtttctcatgcatcatatgtctcattagtgcgttaaatctccagccctgggtgtgatttttagtattacgatgtgattattataaggaaaacttggtgaaaggtcagtgctggagtccatcttgtcttcagccaactgcatgtggtctggttcttatcaggaatgccagaggcctgcttttagcaaccttgggagacagcactcaaggacataaatggttagtttcttatttttatggttacaaactcagcctggtcagctaacttaggagagaggtccccctttgctatgtgacgtgggtcagcttgttaacggatacaagaaggcagcagagggaatatgcctggacatgtgaggcccagggggtcttggtgacccagtctcttgtcttctctatactgtcttagcttggcttaggtcaggacatggggctgtcatggagagcctctctctgctcaggaatggtggggctggggtttgggggcagggtggctcggtttgtctaacctggcttgtcttctaagaaggtggtaagcactatgtctcaggggagtgagcctccccctcacaaaagctttccatctacattcatgggcatgtcagtggtgtgacatgccatctgagtgactccactttgctggtcattttatgcccagcagtgcttatacagtccagcagttcacactggaaaatacggaaatcacaggactgctccatctgtaagcattatgttcacaggttagtgtcacaagatgaaggggcaggagaagatgctaggggccagtccctagtatggcacgacagagcccctgcccttaccagcatgtggatcagccagaaagatggtggggggctggagttcctcaccctggctctccagagcctcttgtgtgcctgtcttccagcttgatggtgacatcccattagtggcttgaaactgaccatggtgacagtagttattttattttatttatttattttttgagacggagttttgctcttgttgcccaggttggggtgcaatggcatgatctcggctcactgcaacctccacctcccaggttcaagtgattctcctgcctcagcctcccgagtagctgggattacaggcatgtggcaccacacccggcttttttttttttttttttttttttttttttttgtatttttagtagagacagggtttctccatgttggtcaggctggtcttgaacttttgacctcaggtgatccgcctgccttggcctcccaaagtgctgggattacaggcgtgagccactgcacctggctggcagtatttctttgaaactgacgatggtgacggtatttagtgggaaattggcccacactacagatcagggctttgtttaccagtacagcactagctgtgaacaagtgcggtgtgatgacgaaagtgctcaccaggtatagggtggcacagcaggggctgggactgactttattgcgtggagctaaggaggcggggcttacacagtattgaaggatgagctgtgaagacagagtggtaaagggaggcccagacaagtgtgggatggcaagacgtatcctgtggttctgtgaaaggaaactgtggatgtgaaggcacttggcaacttaatacaaggtgtcaagcgaaaaggtgtagagtaagcaaatccaacataggatgtataaagtgaactaattagggctggggaagggtatccatgtcataacacattaacctttgctctttagctgataggatccctagggatttcaaatgtggctcaacaaggcctttgcccaactctttggtaaaggggaacccaggctttggagagggacttggcgtaaggaagcaaataggctagaacactctggggctgccagctaaccgggaaatgttgacaaaggctgtgaaatgccctgtgttttggtggtggggcggtgggagtggcagggaagttggcaggaaacgggactaggaaaacaagtggggaggggtcagatagagcaggctgagcgtgcacttcttcatgcgcctcagagccagacagccccgcctcaaagcttgttttgctgtcccttgtgtcgggtggggtcctgggtgccatgggttgcttggtgagcccccgttggtccctgtgccccagactggggcgagtgtgtcactggactgtttccacagctcggttggtgccacactgtcccgaaggttctgaaggagactgaggtcaccaggctgtctgggcagacagagctgccagactctcgtccctccctttgatgggcctgttatcagggttgttttgctcctggagctgatcacaggccacgtttcccagccagccacccccatcagcccagagccccctgggacacaggcagacgtaggcagtcagtttggagagactgcaattcaggaaagggataggctgaaatttcttgggccaagaaattggcccaggactgaccttggctaagcccccactgatgacagacggattttttttaaagattattttctttagacattcttgctctgtcgcccaggctggagtgaagtggtgtgatcttgactcactgcaatctctgcttcctaggttcaagcaattcttgtacctcagcctcccgagtagctgggattacaggcacgcacccctatgcctggcttatttttgtatttttagtagagacagggtttcactatgttcatcaggctggtctcgaactcctgacctaaagtgatccgcctaccctggcctcccaaaatgctgggattacaggtctgagcgactacacctagcctgatgactattttgttgttgttgttgtttgtttttttgagacggagtctcactctgttgccaggctgaagtgcagtggtgcaatctgggctcacggcaacctccgcctcccaggttcaagctattctcctatctcagcctcccaagttgctgggactacaggcatgtgccaccatgcctaatttttgtatttttagtagagacggggtttcaccatgtttgctaggatggtcttgatctcttgacctcgtgatccgcgcacctcggcctcccaaagtcagactgttttaagagtaatttgtaaaaggaagctggaagtttggggtgaatgggtggttacacaaatgctgtgtgatttagggtgcagtttacaaaagagtggctgcgtcctaggctgcaaattctgaggtgtgggcaagtggggaccttggccacccctgcacagggagatctggcccaaaggcagaccagggtcatgaggctaggcccttcttaagagggtggagggcttttggaggtgctgagggcatgaggtcagagtggtgcctgctgctggccttcaagcatgcagggtccctgtttggtggtgggcctcatgaaggagtcacaccaaggtgggtgcttagggttccctcaagcacccattctgtccctctgcatgcaggggaccacacctagggcaggaactgagagctgacgagtgttgtgttcccggcgttgctccttcagctgggatggagcccttggcgggataaaaggctaaactgggtccaggtgagatcaccacaaggtctcacccagggcaatgaagggctcacaagcagcacatccctgctcacagttcgcactcagactcacccgtctgtcgatggtgctgccatcactgctgaacgtttgttctccgtctttctcatgcttttgccaagcagaaggctgagttcacctaacctggacgcaggcaacgagatgcactgcgtgccccccttccccttgtgcagctgtggtgtgaacgcataccctcccttgcttgcccttcctgcctggttgccagctctgctcttgctgttccctgtgcccacggcactctcttgcttattctgtcaagcctgacctaccctcagaggcccaccttggccatccacaaggctccttctgagaactgagccctttccctgagttcccaaagcacgttgtgtgaagctctaaactttctccaggctgaacttagcttactcgtggctgttcacatcctcagggcccacgcagcaaatgctctgtaagggcttgttaaactccactctcctgggactcctagtcccccaccagagtccaccatgactacctcctgcaagagcactgagctcttgggtgccaggcccagctcaggattttcacagacaccaactcgtttaagttgcataaccccgggaggaaggggttgttacctgatgagcaaacaaatcagagaagttcaatgacttgtccaaggttacagaggcttggagctctggattcaaacccaagtcactgtgactcaagaccagtgtttttccccgttacaagcagacacaaaagaataacaatcctttatcatttatactgtattctcacgttacattatttcatttgatcctcacaacagccctgtgaggtaggtagggttatgtattactgttatgcctatttcacagatgaagaaatggaggtggctcagtgaggtgagtgacttgcataaagtcacacagcatgttagtggcttcactgcttctgcaacttctctaaagtcaagctgcttggattagggagcaccgaaaagatggacagttttatggaaaaggagaaagggcagtggtgatttgtaagatttcttcctcaaattgttttgttatgaccacagcttctgctacttggtctaaactaggttctccatgaggggctctcccaagtacccttggcctgggacagctgtctttttttgggatggagtctctctgcaatggcacactctcagctcactgcaacctctgctatctgggttcaagcgattctactgcctcagcctcccaaatagctggggttataggcgcctgtcaccacacccagctaatttttgtatttttagcagagacgacgtttcaccatgttggccaggctggtcttgaactcctgacctcaagtgatcttcccgcctcggcctcccagagtgttgggattacaggcatgagccactgtgcctggcagaaaagctgtcagagagactcctgggtcccaccgtagggggaaaagggcctttactgctcaagggaactggcgctgcccatggcccagtgtacaagctccctactcacagggcctatctatgggaagcccctttctgggcccacctcccatgggtgcctgtgtcaccaggcttccttggctttgaggacccctgcccagtgggggaggaccctggggactctcaaggccaaactcactttagggatgaatcatgacgtgcagtttggaggcctgcatggctcatcacccggtgggaagcagtgcttgggttgcttgccctggatggcaccctacagagacccaggggtgggacacaggactgcaggggaaggccgccttcctgagggcagggtcttaggggccgcacatttactctaagggcactgttacggttcataccactcctgggctccttgctgcacagcacccttggagaccgtctctttcctctggatctcctcaggggccgtgaatcttcctcttccagcagaaaatggtcgaaaggtatatggagccacagatggtggggaaggtgggtgaagtgggggtcacaaacgaggtgaagctacaacaaaaggggactgacttacaggggatagaaaagagctaactttgattctgaagacagatcccagaggggaatctgaattctgagtaatgatgttttcagaaaaatgtctggtctctcaaggtgactattttgaaagcaaagatatataactgggtgaatgggaaggtctgcaaccaagatccagcaagtggcaggacagattttggctcaatatagggaagaactgtctaacagtcagcactgtccaaccacagcaggagctgccttggggatggcgcgcttcctatcagccaaggtatgcaagcaggccaaggtgatcacttgcaggtacccagggagaggcgctcatcttcagatgtgctggggtgttctaggggtgggggaggactaagcaatcatcgaattatttccaaccctgagaatccgtaatagatttgtttaaaatgacctgtttcaatgatacctcaaatttttgggaaaacaatgacaaaagtcaggtaacaggctggatttggaagtgatttcattacactggctacagatacttgcacatttttgcaaagatagcagaccctttctaaagccaggaatcccttgattcagccctatcaagagggacattcctaccttctgaactctgcctggacactcattcctctagaccctgccacacctcatctctgacccccaagaggggacactaacagagctctgagaatgcacagcattccaagccagccaaacatggtcacccctgggcaaacatccacgatcatggatgcaccctggggagggaggagcaggtgtgacaggaaccgctggggactttatgtcctggttaactggaggtagcctcttgttctcatcccatcctctccaccagggccttcttacagatggataaatgcggcactggctagcggtacagagagatggagcccaggagaaaactgctgctcttttgtcctggctggaaaatcctcctatggcctcctttagtacacatcagctgactcttcacttgtcataataaaccgcctgtctgcagctcaaaatggcaagaaaaaaggtggttctgttcagaggacactggattccaaaaggtggacttcatcatggggggaatcaagtcctgctacacaattaatacagattagtatagtcccctgcctcgcaccactggcgagatgccctccaagacctggctgacatgagcttcctgggactccccatctccagtgttgttggggtccttttacctgtaccgtattaatcgaataatcttattgttcataaaatccagagcatgtgggtgagaagaatcgatgcagaagccatcgctcagagcgattttcagtacctcttccacgaagacctggaagctgttgatctgcttgttggcgggcaccacccagagcctcttgaggttctgcttggtgtactcctcctctggcaggccctccacattggccacccagtccagagtcaggtggttggggtcctgcaagtggctggtgatggaggagagattgccattggagcagtagaagagcttagagccaaggagcttgaggaagaggcaggaggtgctgaagatgttggcgttgaagacctccatgctggaagaggagaggctgtagatctggggtgcctcgcgcacagtgaagtggaccgtctgcacacgctggttcagtgtgatgttgagcatggcattcttctcggccttggagagctccacttccttctcctgcagggcctcaatcaggggctgcacctggtagaagtcggcctccctgcggagcagccccatctcctggaagtcctcaggcaggtcaaggtgggaggtccgcaggaagttgaggatatagcggaacactttgccgtcacggtcaatgaagcagttgccctggctgtccctcttggtgggcatcttcccgctgaacatggcgcctagcatggagtcagggaagctggtcagggtcgccagtgaggttgtatagagcttccccccgacgttcagcgtgatggggtcggacatggcaggagactgggttgctggaagtagtcctggagagggtgagaagtgagaaatgactataaccaaaccttatcagagaactcacagtccttaactccttattgtacatcaagcctatgcccttgggatcgaaatgaattaatacctgatgaatgggtgataattgctaacactgtgcaaagtacattagcttcactagggagagggaaaggagaaagggaaaaggaaaggaaaaaggaaacagggaaagaaagggaaggacccctaaagcctgggtcccccgcgcccccctctgctgccgacattctattttaattggtctggaatactgccccagccctggtattttgaaagagttcccttagtgattctaaatgttcagttagaatagagaaccactggtctaaagaaagaaaggaaaacgtgcctggactgaaagctatgctacgtgtttatacatatatttgcttacataatagttgtatcagccaggtgcggtggctcacacctgtaatcccagcacttagggaggccgaggcgggcagatcacctgagatcaggagttcaagatcagactggccaacatggtgaaaccccatctctactaaaaatacaaaaattagctgggcatggtggtgggtgcctgtaatcccagctgttcaggaggctggggcaggagaatcatttgaactcaggaggtggaggttgcagtgagccgggactgaacaactgcactccagcctggacaacaagagtgaaactctgtctcaaaaaaaaaaaaaaaaaaaaggttgtatcatcttttgacacagacaatataatcatccattcctgttgtgcagatgaggaaaactgtgccaggattcattcctcaagctcagaaaggtgaagtgccttgccaggattcaaactcaggtccttttatatgaaagaaaaatgtgtaaatgcttactagccaaagatcggttctaagtactcaacaaatggtttgagatttatgactccaggctcagtgctcctccttctgtaccagtgacactgctgaatttaattattattctctctcaaagatttgacaatgaatggccatgattagtgtcatttccatctatcttcaaggcccacagtgggtcactcagctgtccggactgcaaatgacaatcctgttttttttaaatttttttaaattttattattattaaagttttagggtacatgtgcacaatgtgcaggttagttacatatgtatacatgcgccatgctggtgtgctgcacccattaactcgtcatttagcattaggtatatctcctaatgctatccctctcccctccccctaccccacaacagtccccagagtgtgatgttccccttcctgtgtccatgtgttctcattgttcaattcccacctatgagtgagaacatgcggtgtttggttttttgtccttgcagtagtctttactgagaatgattatttccaatttcatccatgtccctacaaaggacatgaactcatcattttttatggctgcatagtattccatggtgtatatgtgccacattttcttaatccagtctatcattgttggacatttgggttggttccaagtctttgctactgtgaatagtgccgcaataaacatacgtgtgcatgtgaatgacaatcctttctagactttcactgctcccatgctgacttgccttgccatgaagatgctcttggctcctggtggcctgtcctttgctgtcataacaccccaagcttctctgaaactggctgcaaggtcagtgcaaatgcaggactctgtcagagcactctcctgaaggcaccctatccactccttaacaaagcccccctaatctctcagctgtcagcatctatggctggcaagtctccttcaccacatgttctagaaaacaggagatctcagctccagcttccaacactatactttccccagagccaacctgtggaccagattcagcaatccttccctgacatggcaggtagggatgccaagaggaactggccccagtattacccccagaagagcctgtgctggtagaggagatgagagagtaaaggcacagtgacaagacaggtagaagatgtgggagatggtctggaatgaaccttggcacatcaaaaactatctggctggcagacacagtggctcatgcctgtaattccagcactttgggaggccgaggtgggcagatcgtttgagctcaggagttcaaggccagcctggccaacatgacgaaatcctgtctctacaacaacaacaacaacaacaacaacaacaacaacaaagcagggtgtggtggcgagcacctgtagtcccaactacttgggaggctgaggtgggaggatcgcttgagtctgggaggttgaggctgcagtgagccgtgattacgccacagaactccagcctggcagacagagggagatccggtctcaaaaaacaaacaaaaacccaacctttcaagcatgcttaccgtccagatggagaaacagaggtctgaggagacacctccccaaagtcacccagcaggggtggcgagaacttctcaacatttctgcctcctgaagaggctgactgcactgaggcctcctttctccagaggatgggggccatgccagtgtgagagatattcagctgccatgctcgactctgtggcaggggcaggggtatgtcccctcatgattcctccccagtctaatggggcactttgatattgcagaaaggcattccccatctggagtgtctgaggagaaatgagtccatgtgaaatacgaaatcgtcagtagaagcctggagcagatgggctgcgagacggtggggaggtcccacagaggtgtgggcgagctgccgctgggggcatggctgctggtgtggagatggctgtgaggcagcaactggggagggtgctgtgactcagggctgaatctggccaattctctctccagagggcccaggcaggaggtggacccaggagtgggtcccctttactcagtccagaatcttcttccctgttgccctctttgtaggactctagtctactccaactgttttcttctggatagaaatgtgcatgttctagtttcagtctctctttcttcttgaaaacaaacactccctcccaacattgggtttcaggcctaggatctataaagtggcttaccttcacggcaggaaaagaagcgggtgtcagagaggacttggacagttccatgggtcacagacccttatctcttccatgcattactgctgaagtgctcagagcattacaggcgggggaggtcaggagccacagggcattccctgggacccctgggtaggtgcccaaactcttatttctctgcttgcaaaagcagcagagcacctgctttcctgcgcccccgatgacgtcatggctcccggtcatttgtctggggagagcagcacagttgcacaggcagtcagggcagaggctggctagcaggcaggtctcctgctccgagtctagggtgctctctgtggtgatctccccttacctatccagtcacagagctcagtcctctaagcccttcttttctttttttttttttgagatggagtctcgctctgtcgccaggctagagtgcagtgacgcaatctcagctcactgcaacctctgcctcccagattcaagcgacactcctgcctcagcctcccgggtagctgcaattacaggtgcacgccaccaggcccggctaatttatttttgtatttttagtagagatgggatttcaccatgttggccaggatggtctcgatctcttgactttgtgatctgcccgcctcggcctcccaaagtgctgggattacaggcgtgagccaccgtgcctggtctaagcccctattttctagccacagggccacagtgtccatcatcacaaatccacggccctgctacaaaaggcaaaatgacatgacacgaggggagtttcatgagtcggcggaggtttgcagtcaggtagtaatgaactaacaccactgagcctcagtcccctatgtgttcccagatgcctcctggccacctccacttggatattctgcaagtgaggcaggagaacaaggtttggagacaaggaacttaaggccaatttgtgctaacttcataaaagagaaaacaccaaggtctggggcaaaaaatctgaggccaattctgttgacttcccaaagctggatcagaaggaaaatcctgggtctgggggcagggaccctaagaccaattaacaccaaattcctaaagctgaaccaagaggaaaaacgccatcgccccagctgggtgacaaaggatcaaacgctcctctttgcaaccctcccactccaccatgtctcaggtggaaagggaggggggccttggattgggaccatccctttatctgcatagggtgcccattcacctcagcctttaattagccacagactaaatccttcgtccagataaggagtagctgataagaacctcaaatggggtacctaaagcccagagaactttgtaactgggcctttgagttccttgctccggcccactcccaccctgaggagtgctttctcactttaattcctgctttcactgcttcattcctgcatttcattcccctgctactctttttatttttactttttttttttttagtagagacgggtttcaccacgttggccaggccggtcttggactgacctcaagtgatctgcccatccaggcctcccaaagtgtggggattaccacgtctgcccctctgctactttgttcaaaacgccaaggacctggaaaattcctagtcaagaccctccaccagtaacacaggtaccagaagcctcacctgtgccttcctctgcccctaactctgctgtgaactcttctgagccacccaactgcctgagggaagcagccccgactcttcagccctacagtttgtcttgactatctccttgaacccatccctatagctactcccagagctcaggcctcatccctgccccctgcacccctacatcctcactcggctccctgccttctgtccaccttcctttctggtctgcagactgaccttcctgaagtaccgtccctgcccaaccaccttcctctttccctctcatgtctaaccctgccctccacgcacgccagacgccactgagtcagtctccttcatgctgacacctgccctgcctttgctgtccctagaccacccctgtctaccttctctaccctctctgctgtctacctttcaggcccagcctatgagtcatcacctctgtactactgtccccagatctccactaggttaacagctctcctctcagggcttcctggtgccttgcacaaccccctctggcagcacccagcattccacctgcagcgatctcctttgcacatggccctcttccctggctatctcaaaatgcagatcagtggacatccatatggggaaaaaaagaaccttggtccatctcttatgctgtttatgaaaaataaactcagaacatatcatagacctaaatataaaatctaaaactacacaacttctaggagaaaatctttgcaactgtggattaggcaaagatttcttggataggacctaaaagggacaaaccattaaaaacatcaataaactagacttcatccacgtaaaaaacttctgctcttcaaaagacactgttaagaaaatgaaagaataagacacagatggagaaaatatttgcaaaacacctatctgataaaggattgttaaccagaatatataaagaactctcaaaactcagtaagaaaataacccaattaaaaaacacataaagctgggtgcagtggcacatgcctgtaatcccagcactttgggaggctgaggcgggcagatcacttgaggccaggagttcaagaacagcctggccgacatggtgaaaccctatctctacttaaacaaaaacaaaaacaaaacaactagacaaaagatctaacaaatactccagcaaagaagatatgcagatgactgacaatcacatgaaaagatggtcaatatcatcagtcatgagagtatgaaaattgaaaccacaattatatgctactatatacctattagtggctcatataaaaactgacaatgccaattgaaggtgagtacatagaacaatggaaacctcatacactgctgacaaatggtaaccgctgttgaaaatagtttgacatatggtatggtttggctctgtgtccccacccaaatctcaccttgaattgtaataatccccatgtgtcaagggcaggactaggtggagatgattgaatcattggggcggttttcctcatgctgttcttgtgatagtgagttcttaagagatctgatggttttataaggggtttcccccttcgcttggcacttctccttgctgctgccatgtgaagaaagacatgtttgcttccccttccaccattattttaagtttcctgaggcctccctagtcctgtggaaccgtgagtcaattaaacctctttcctttgtaaattagccagtctggggtatgttgttattagcagtgtgagaatggactaatacagcatacaattatcacgtgacccaatgaccctactcctaggtatttatgccggagaaatggagacatatgtccatgcaaagactcatatgtgaatgtttatggcagcttattcataattgcccaaactcagaaacaactaattgtaaacagataaattgcagtacatccatacaatagaatatgactcagcaatatataggaactactaaaagacattaccacatacatgaatttcaaaagcattacgtcaagtgaaagaagccagcctcagaaggttacatggtacatgactctatctgtataacgttctggcaaaggcaaaactataaagacagaaatcagattaagtgaattcttggggctgggttggagggaaaagactggttacaaagaaacacaaggcatattttggagtgatgggaatattctattaggttggcacaaaagtaatatgatgtggtggtggtcaaatgtgtttataaagactgtcaaaattcatgtaaccatacacatctaaaagggtgaattgtggccagctgtggtggttcatgcctataatcccagtactttgagaggccgaggcaggagggtcacttgagcccaggagttcaagaccagcctggaaaacatagtaagacccctgtctcaattaaaaaaaaagtgtaaattttaccatacttaagttttacctccataaacaggaaaaaaaatgaaggttgtgacctattggcaagtcatgacatcaatacagttaagtgggttgtaaccaattatatattgttgtgaaacttttgtttcagttgtgtgtctatgaactggatcatggtatcttcatactgtggctgacaaaacaattttttttggaaaccactgcctagtttgtgagctgagagcaactgcatgtctcacgtctgcatagtcaccgccttgcactcaggggaggggttgtgtgatactgtgatataataagcaatatacattttagatttgtccccagctgttggccagggctaaaaactgtgtaatttcccaagtgataatagctataggaacttcttctattttaataattggttttgtcccagtaggtggaggttgcagtgagccaagactgtgccactgcactctggtttgggtgacagagccaggccttatcccaaagcaccccccccccctcaaaattaattatagttgttatgctatcctaatccaaatcccaatagtatttttcaaaaaagatttaaataattaaaattttaatctggaagcactgatgactgataacctgaaagagtatttgaataagacatcttagcgcaatgtattaaactagattataaagcaatgacatttccctctgtggttttaaaacaatgagaagagaaggaatttggtaataaatgtgaagagacacaactagcaatgtttaaaaataaacatattgataaatatattgatccttacaactctgggacccaggcaggaccgattacattgtctttgtttcaaagacaacctaaagcagacccaaagagcgtgaatcccttgcccagggcttcatgtggattcttgacctcagagtcatggcctctgggaagttctggtttggacacccttcagagaaccagtcctttagacgtcttcggcaggaagctgaacccagccacagaggtcctagccaacagtctgtatactcatggcgacctggagtaggcaagagctcttttctgtgcaatttccttttttcctacattactgtccacttccataatgagcattacttccataatgaaatgacttaccatgcaatcctgtcagtggggactagctgttgtaacaataaatgtactgagtaatgaaaattgtccatctatcatctatctatcaatcacctatccatcatttcctagctctgctcactcacagagcctagaaatgacacccagtagcaataaacacacctagccctggatctttgtttctgaatgtcatcatctcttctaaaagaaaccagggcttcctggagaaactgatgattcctggactttcacaggaaaagttcaaggtgagttcatatagcccagtgaattaaaaaacaacaacaacaaaacaagagtccaggctgggtgcggtagttcatgcctgtaatctcagcactttgggaggccgaggcgggcggatcacctgaggtctggagttcaagaccagcctggctaacatggcaaaaccccatctctactaaaaatacaaaaaatagctgggcggggtggcgtgcgcctgttattccagctacttgggaggctgaggcaggagaatcgcttgaatctgggaagtggaggttgcagtgagccaagatcgtgccactgcacaccagccagggcaacagagtgagactccatctcaaaaacaaacaaacaaaaaacaagagtccataatgttattcccaaaagaaaaaagagggtgagaagaagggagtgagaaagatagagacaggaaaaattaattctttttttttttttttgagacggagtctcactgtcgcccaggctggagtgcagtggtgtgatttcggctcactgcaacctccgcctcccagggttcacgccattctcctgcctcagcctcccgagtagctgagactacaggcgcccgccacctcgctcggctaattttttgtatttttagtggagacagggtttcaccttgttagccaggatggtctcgatctcctgacctcgtgatccgcccgcctcggcctcccaaagtgctgggattacaggcgtgagccaccgcacccggccaggaaaaattaattctataaaaaagaacactaataaacacagaaggaatggcagaattagaaaattaccaatttgcaaccctcagtgttgtaattcaagcaaggatcatccaaggatgtggtgtgaaaggttatcaggaacacataattataacagactcagagaatcacttcacagattacttattaattgtaagagagaaaaatttacctttacgatagagagatcaggttgccatgaccttaataatcaaatttagcattcatggatagtaagaccctgacatcatatcctcctgacatgcagtaagtacacacattgccaaaaacttaaatctaaccccaatctaatcattgataaaccaatcaaacatatttaaataggtggagaagctctatttatttatttatttatttatttgagatagagtcacactctgttgcccaggctggagtgcagtggcacaatctcggctcactgcaacctccacctcctgagttcaagcaattctcttgcctcagcctcctgagtagctgggagtataggcacacaccaccatgtccggctaatttttgtatttttacttgagatggggtatcaccatgttggccaggctggtctcgaactcctgacctcaagtgatctgcccacctcagcctcccaaaatgttgggattacagacatgagctaccatgccaaggggctctatttagattaaatgagttgataaaagagatataataggatgcaatatgtacatcttgactggatctgattttaaaaaccagaaactatataaggtatttttgggcaattagaattttttttttcttgagacagggtctcactctgtcgcccaggctggagtgcagtggcgcgatcatggttcactgcagccttgacatccccaggctcaggtgattttcccaagtagctgggactacaggtgcacgctaccacaccatgctaaattttgtagtttttgtagagatggggtttcactatgttgcccaggctggccttgaactcctgggctcaagcaatctgtctgcctttgcttcccaaagtgctggggttatagacatgacccattgcacctggcagaaaattttgaataactattacattgggtaatactgacttcttgcttattacatataatgtattatggctatgtagtagaacgcccttattaggggatacatgttgaagaatttaggggtcaaacgtcaatcaacatatctgtaacttacttttaaataattgggccaaaaaagtgtgtgtttgtgtaaaagcacaattggcaaattgtgatttggcgtatctaggttaagggttgtactttaagcttttatgtaagcttgaaaattttaaaaatatttgtattttaaaagaggggggaagaacacttcttcatagggccaatagatgtaaagttacagtcattgtgccagaccccaataagtcgccagatttagtaagttacagatttactactactacttagaagtgtgaaaagctcataattttccttcccaagtgtttctgaattttgaagggtgaggatgccaaccagggctaagtggtggaggagttagaatctaagcctgggtgggcagtctggatagagccaccacccacagaaaccacaccactaggcctattttttttttttgagatagagtctcgctctgtcacccagtttggagtgcagtggcgtgatcctggctcactgcgacctctgcctcccaggttcaagtgattctcctgcctcagcctcctgagtagctgggatttcaggtgcgtgccaccatgcccggctaatttttgtatttttagtagagacggggtttcaccacgttggccaggctggtctcaagctcctgacctcaaatgatccacccacctcagcctcccaaagtgctgggattacaggcgtgagccaccgcgcccggccaggtctgcctatcttctgtgctgtgattctgcctggaagaagatggctgttcaggccttgtttttcctgcctcatcatgaacccagaatctctgtaactcggcaggaaagaaactcctgtcatgactcactgtgaatgttgacaaagagaggccactcctgggcctcagaaatgtcaaggatgggcagaaagaactggagacaggccaggctcagtggctcatgcctgtaatcccagcacttggaaggcccaggtgggaggatcgcttgagttcaggagtttgagaccacctggacaacataatgagacctcgtttctatagagaaaaaaaaaaattagctgggcatggtaacacgtgcttgcagtcccagctgcacaggctgaggtaggaggatcgcttgagcctgggaggtcaggctgcagcaagccgagatcacgccactgcactccagcttgggcaacagagtaagaccctgtctcaaaaaaaaaaaaaaaaaaaaaaaaaaaaaaaaaaaagaaaagaactggagaggctgtgtggggtaggggcagaccatgcatggtattcggcattacctgggtttgcatttgaatcatcccctgccacttactagctgtgcagccctgggcacgttcctgggcagatgatgatgatcataacaacaacaaacactacaacggcaataataatggctaacgtttattgagcagttactaccgtgctaggcactctatacatgttaatctctttcaatctcacagattaagtagatactattatatacctactttacagaccaaccatactgggccctcttcgacttctgcaatttggcatctgccatcccctctgcctggaccttgaagacaaatcccatgcatcggccaggtctctcagctggaattcattccttctcctctgcatctgcggagaattttagtctaaccccttaccacagccaactttacttaagtagatggatgcctggctcccttgctggggctgggggtgcccctttctatatccacagaagcacgtagaataggggaagaggaagaaatttctctcctgtttagtcagatccattttccccacctggattttcaaaactttttaattttgaaatgctctgttcaattttcaatctggaatctaaagggatgtgacaaggccgaggtgattacttgggagtaaatgcaaggagagtttccactgcgtaagggaatggacagcacgtggctgggtaaatttcaaaaaagacaatgctcagagtgagtactgtgttttccagtaagtgggagacttccccaccttaccccaccggagaaaggactctttgccttgaagcccctaggacgactaccctttcttcacctcctcttcctgaccaccccacatcaaagagctgcgtctataattctgtcttgcccagtccatatctctaagaaatccgaaggctgctctattaccatcacaacccatagggaggagagaaaaggtgcttcctagacacagggaccccagaatgagctgcgcaaaaggcaggaggagagaaaggtgggctggaagaaggagaccacggaatctggcacggcaaggtattctttcatctggcgtagacctccaacaaccactaccatcccccagccccttccagaactttccttcaccttctcggctcactctcacgcttcaccttgtgcttttcctccggcaccagcaccccatttcaaaccgctctcttcctatgtctcctccaaacagtaatttataaacaccgtgctttcaaatccatctcatttcatcctcacagtactataggagggctgccttacaggctgtcattcggaagggttggggggactggtcctgccagacaatttgcgaaggtcggggcatcaaaacttttctttcctgcctcccacgccaagctaaacctctgcctccccgagtgcagggagaaaagcaaggtctttgaagtaaggagtcctgagtggttttaggcaagtcccttcgcttttctgagcctcacattcctcacctgtcaaccgctatagtgacgcctctacttttcagggcgtgggaggtcaatgagatcactggtgtgccgcactgaactcggggatggactctttatttccagtcagtaaagggaggtgttctatgcatcacaggcttttgcccacgccgccacagcgcggccggcccagggctctccagagcggggcaagttccctgtcatctgtgtctccgcccttccttccccaatctcacccaggctggcctcatcggcttgttccgcacccactccccagccctacagcccccactccgacctgacaagtgccccacccccacctagagatcccgcctctccttgccccacccaccaaggactcctcccacagtcccgaccctcattatccgggcttttccgactatcaccatccgcccttgctctcccaacagccccgcccctctaagagctctgcccacttcggctcacctcgctccgaaacgcgccccctcccccgcaccacaggcgatgccatccccatttcacagatgggaatggcgaggctgggatagattagtctcatggtaagacaggtgctcagtgggcccacgtaccctcgccggtagtccccgagccccgcgaccccggccgtgccgcacccacctcctgccctcgctctgcagcctctccctccgcgagcggccagcgcagctttgcgaggggggcggagggtaggagcccagcccaggggcgtggcggtccggggtctgtgcgctgccttccgggactggtagttctctctgggctcctcgcagctcggcgcgtcgagagcgaactccatctcccaggaggcattgcagcgagttgctctgcgccccgcctcgtccccgccggtgtccggccggctggccttaaaggggacgcggtcagagtggcagctcgcggggtggcccccgctccggctccgccccatttcacgcacctcgcccctccccatgcttcatcccgcagccccggggccgcgccgcagagtcgggcttcctggatacatagaggcttgtgccaggcggggtgggaagactggattttgcagtggaagcagcatctcttccgtctgggacctggctgagggcgtccccaccctggggggagcagaggaccagccctgacctagccgggcccagcctcgtcctgcccggcctgagcgccccgcccagcagccggctctggcccaccggggtccgggaggtaagggggttgggggcgtgtgactgtctttggggggcggtgtctgcaccctggcctttcgagaaccctggacttgcgagcccagggctgggagtcgggagatctgggtgcatcctgggcctcctgggcctcactgtgtgaccttgggcgagtagctattcctctctgggcattaatcccctcccttcatcaattgtatgtcgaagtgatcctgctccctcccctttcaggtaagagggcattcgcacccagtacctagtacattcgcttgctggcttttcgtggggccctccagctcaagcgtgggctgggcgcgggtgcactggagggagggaggtggcacaaactggggggcccgaggccaccattgcttggaatgggcttttcctattctcagtgtgctgggccctgggataccctccccaaccccctcttacggccctggcccttgcaaaccggagaccctgatgtgtcagcttggtgcccttccttccagaaagcttggggtggagtgcggccctcacaggtttgtgaggggtggccccgcagggataggtgtgtgtggtgggttcctgaggaggaatagctgctcctgctttcttcccttctctctcctccttatcctttgaggaagagcagctgctttttaccagtgaaacccttttttctttttctttccaggagacagtttggtggcccagacatgggcctcccggagtagggtgaagcacttcttcccagggttgccttgggtagaagttcagttaaaaaatacctagagagatgctgccattcagacacctgaggggccagagatggagggcaccacctggtgtcgggaggggcagcctggtacccccaaggaagctctggctcccaggggagtcaaaggagggaggctggcttcgagggtcagagaggggaggaaatgtgctcctcagctcttaggtgctgagcagaggctgggaggggctgctctgacctggagcagggagaggttgagtaacaccatgaagccttcttctcaggtcctaggagattctgtgagcttttgacctgaaggctaaccaggaacagatttttgttttgttttgttttgtttttgttttgtgctttttcttttgctttggttgtttgaa

SEQ ID NO: 2 - protein, 339 aa, Homo sapiens
MSDPITLNVGGKLYTTSLATLTSFPDSMLGAMFSGKMPTKRDSQGNCFIDRDGKVFRYILNFLRTSHLDLPEDFQEMGLLRREADFYQVQPLIEALQEKEVELSKAEKNAMLNITLNQRVQTVHFTVREAPQIYSLSSSSMEVFNANIFSTSCLFLKLLGSKLFYCSNGNLSSITSHLQDPNHLTLDWVANVEGLPEEEYTKQNLKRLWVVPANKQINSFQVFVEEVLKIALSDGFCIDSSHPHALDFMNNKIIRLIRYSNHLTLDWVANVEGLPEEEYTKQNLKRLWVVPANKQINSFQVFVEEVLKIALSDGFCIDSSHPHALDFMNNKIIRLIRYR

SEQ ID NO: 3 - DNA, 783 bp, Artificial Sequence
  Synthetic construct
atgtccgaccccatcacgctgaacgtcggggggaagctctatacaacctcactggcgaccctgaccagcttccctgactccatgctaggcgccatgttcagcgggaagatgcccaccaagagggacagccagggcaactgcttcattgaccgtgacggcaaagtgttccgctatatcctcaacttcctgcggacctcccaccttgacctgcctgaggacttccaggagatggggctgctccgcagggaggccgacttctaccaggtgcagcccctgattgaggccctgcaggagaaggaagtggagctctccaaggccgagaagaatgccatgctcaacatcacactgaaccagcgtgtgcagacggtccacttcactgtgcgcgaggcaccccagatctacagcctctcctcttccagcatggaggtcttcaacgccaacatcttcagcacctcctgcctcttcctcaagctccttggctctaagctcttctactgctccaatggcaatctctcctccatcaccagccacttgcaggaccccaaccacctgactctggactgggtggccaatgtggagggcctgccagaggaggagtacaccaagcagaacctcaagaggctctgggtggtgcccgccaacaagcagatcaacagcttccaggtcttcgtggaagaggtactgaaaatcgctctgagcgatggcttctgcatcgattcttctcacccacatgctctggattttatgaacaataagattattcgattaatacggtacaggtag

SEQ ID NO: 4 - DNA, 601 bp, Homo sapiens
atttttagcagagacgacgtttcaccatgttggccaggctggtcttgaactcctgacctcaagtgatcttcccgcctaggcctcccagagtgttgggattacaggcatgagccactgtgcctggcagaaaagctgtcagagagactcctgggtcccaccgtagggggaaaagggcctttactgctcaagggaactggcgctgcccatggcccagtgtacaagctccctactcacagggcctatctatgggaagcccctttctgggcccacctcccatgggtgcctgtgtcaccaggcttcyttggctttgaggacccctgcccagtgggggaggaccctggggactctcaaggccaaactcactttagggatgaatcatgacgtgcagtttggaggcctgcatggctcatcacccggtgggaagcagtgcttgggttgcttgccctggatggcaccctacagagacccaggggtgggacacaggactgcaggggaaggccgccttcctgagggcagggtcttaggggccgcacatttactctaagggcactgttacggttcataccactcctgggctccttgctgcacagcacccttggag

SEQ ID NO: 5 - DNA, 583 bp, Homo sapiens
ccccggggttgactaggaattttcaggtccttggcgttttgaacaaagtagcagaggggcagacgtggtaatccccacactttgggaggcctggatgggcagatcacttgaggtcagtccragaccggcctggccaacgtggtgaaacccgtctctactaaaaaaaaaaaaggtaaaaataaaaagagtagcaggggaatgaaatgcaggaatgaagcagggaaagcaggaattaaagggaaaaagccctcctcagggggggagggggccggagcaaggaactcaaaggcccagttacaaagttttttgggctttaggaaccccatttgaggtttttatcagctactccttatttggacaaaggatttaatctggggttaattaaagggtggggggaaggggccccctttgcaaataaagggatggtcccaatccaaggcccccttccttttccacctgaaaaatgggggaggggaagggttgcaaaaaggagcgtttgatcttttgtcacccagttggggggaaggcgtttttccttttggttcaactttaggaatttgggggtaattggccctttaagggg

SEQ ID NO: 6 - DNA, 884 bp, Homo sapiens
  misc_feature (558)..(578): Represents location of insertion/deletion.
ctgaaactgtaattttccctatctataaagtgggcataatatttatatctacctcttaagattgttgtgaaaattaagagttcatccatggaaagaatttagcacaatactctgagtaagtgctgggtaaatgtttctattattacgtttcccagaaaagctgagtcccccaggtatgatttggaaggagaggaagagcctgtttctggcttctaactggagcctatgcttcccagcctgtcagaatggccaccagcaggctgtaaccttttgatttggaaggagaaagagaagagtgagaggggagatatatgtagctaagcaacggatgggggaaaaaaagccagctagtaagaaattctaggtggtagtgttggccaattaataataataataggccaggcatggtggtagtgttggccaattaataataataataggccaggcatggtggctcacacctgtaatcccagcactttgggaggccaaagtgtgcggattgtttgagcccaggactcaagaccagcctgggtaacatggtraaaccctgtctctacaaatatacaaaaattagctgggtgtggtggcatgcacctgtagtcccagctacttgggaggctgaggtgggaggatcacttaagcgtgggaggttgaggctgcagcaagccatgatctcaccattgcactccagcctgggcaacagagttgagaccccatctcaaaaaataacaataaaataataatagctaacagttacattctgcttactatgtgccaggcaattctaggtgttttccatataataactcaattaccaaaacaatactgtgagatatataatttttctcattttatagattaaagaaattgaatcacagatagat

SEQ ID NO: 7 - DNA, 855 bp, Artificial Sequence
  Synthetic construct
atgtccgaccccatcacgctgaacgtcggggggaagctctatacaacctcactggcgaccctgaccagcttccctgactccatgctaggcgccatgttcagcgggaagatgcccaccaagagggacagccagggcaactgcttcattgaccgtgacggcaaagtgttccgctatatcctcaacttcctgcggacctcccaccttgacctgcctgaggacttccaggagatggggctgctccgcagggaggccgacttctaccaggtgcagcccctgattgaggccctgcaggagaaggaagtggagctctccaaggccgagaagaatgccatgctcaacatcacactgaaccagcgtgtgcagacggtccacttcactgtgcgcgaggcaccccagatctacagcctctcctcttccagcatggaggtcttcaacgccaacatcttcagcacctcctgcctcttcctcaagctccttggctctaagctcttctactgctccaatggcaatctctcctccatcaccagccacttgcaggaccccaaccacctgactctggactgggtggccaatgtggagggcctgccagaggaggagtacaccaagcagaacctcaagaggctctgggtggtgcccgccaacaagcagatcaacagcttccaggtcttcgtggaagaggtactgaaaatcgctctgagcgatggcttctgcatcgattcttctcacccacatgctctggattttatgaacaataagattattcgattaatacggtacaggctgtttcagggtccggacccagctttcttgtacaaagtggttgataacgattacaaagatgacgatgacaagtag

SEQ ID NO: 8 - protein, 557 aa, Homo sapiens
MDDLDALLADLESTTSHISKRPVFLSEETPYSYPTGNHTYQEIAVPPPVPPPPSSEALNGTILDPLDQWQPSGSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 9 - protein, 179 aa, Homo sapiens
SPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFH

SEQ ID NO: 10 - DNA, 541 bp, Artificial Sequence
  Synthetic construct
gagcagcccgcagcgcgtcacctccacccaacagcagacacgcatctcggcctcctctgccaccagggagctggacgagctgatggcttcgctgtcggatttcaagttcatggcccaggggaagacagggagcagctcaccccctggggggcccccgaagcccgggagccagctggacagcatgctggggagcctgcagtctgacctgaacaagctgggggtcgccacagtcgccaaaggagtctgcggggcctgcaagaagcccatcgccgggcaggttgtgaccgccatggggaagacgtggcaccccgagcacttcgtctgcacccactgccaggaggagatcggatcccggaacttcttcgagcgggatggacagccctactgtgaaaaggactaccacaacctcttctccccgcgctgctactactgcaacggccccatcctggataaagtggtgacagcccttgaccggacgtggcaccctgaacacttcttctgtgcacagtgtggagccttctttggtcccgaagggttccac

SEQ ID NO: 11 - DNA, 664 bp, Artificial Sequence
  Synthetic construct
gcctaagcggaatgggggccggggcctggaggacgtgcggcccagtgtggagagtctcttggatgaactggagagctccgtgcccagccccgtccctgccatcactgtgaaccagggcgagatgagcagcccgcagcgcgtcacctccacccaacagcagacacgcatctcggcctcctctgccaccagggagctggacgagctgatggcttcgctgtcggatttcaagttcatggcccaggggaagacagggagcagctcaccccctggggggcccccgaagcccgggagccagctggacagcatgctggggagcctgcagtctgacctgaacaagctgggggtcgccacagtcgccaaaggagtctgcggggcctgcaagaagcccatcgccgggcaggttgtgaccgccatggggaagacgtggcaccccgagcacttcgtctgcacccactgccaggaggagatcggatcccggaacttcttcgagcgggatggacagccctactgtgaaaaggactaccacaacctcttctccccgcgctgctactactgcaacggccccatcctggataaagtggtgacagcccttgaccggacgtggcaccctgaacacttcttctgtgcacagtgtggagccttctttggtcccgaagggttccac

SEQ ID NO: 12 - DNA, 1674 bp, Artificial Sequence
  Synthetic construct
atggacgacctcgacgccctgctggcggacttggagtctaccacctcccacatctccaaacggcctgtgttcttgtcggaggagaccccctactcatacccaactggaaaccacacataccaggagattgccgtgccaccccccgtccccccacccccgtccagcgaggccctcaatggcacaatccttgaccccttagaccagtggcagcccagcggctcccgattcatccaccagcagcctcagtcctcatcacctgtgtacggctccagtgccaaaacttccagtgtctccaaccctcaggacagtgttggctctccgtgctcccgagtgggtgaggaggagcacgtctacagcttccccaacaagcagaaatcagctgagccttcacccaccgtaatgagcacgtccctgggcagcaacctttctgaactcgaccgcctgctgctggaactgaacgctgtacagcataacccgccaggcttccctgcagatgaggccaactcaagccccccgcttcctggggccctgagccccctctatggtgtcccagagactaacagccccttgggaggcaaagctgggcccctgacgaaagagaagcctaagcggaatgggggccggggcctggaggacgtgcggcccagtgtggagagtctcttggatgaactggagagctccgtgcccagccccgtccctgccatcactgtgaaccagggcgagatgagcagcccgcagcgcgtcacctccacccaacagcagacacgcatctcggcctcctctgccaccagggagctggacgagctgatggcttcgctgtcggatttcaagttcatggcccaggggaagacagggagcagctcaccccctggggggcccccgaagcccgggagccagctggacagcatgctggggagcctgcagtctgacctgaacaagctgggggtcgccacagtcgccaaaggagtctgcggggcctgcaagaagcccatcgccgggcaggttgtgaccgccatggggaagacgtggcaccccgagcacttcgtctgcacccactgccaggaggagatcggatcccggaacttcttcgagcgggatggacagccctactgtgaaaaggactaccacaacctcttctccccgcgctgctactactgcaacggccccatcctggataaagtggtgacagcccttgaccggacgtggcaccctgaacacttcttctgtgcacagtgtggagccttctttggtcccgaagggttccacgagaaggacggcaaggcctactgtcgcaaggactacttcgacatgttcgcacccaagtgtggcggctgcgcccgggccatcctggagaactatatctcagccctcaacacgctgtggcatcctgagtgctttgtgtgccgggaatgcttcacgccattcgtgaacggcagcttcttcgagcacgacgggcagccctactgtgaggtgcactaccacgagcggcgcggctcgctgtgttctggctgccagaagcccatcaccggccgctgcatcaccgccatggccaagaagttccaccccgagcacttcgtctgtgccttctgcctcaagcagctcaacaagggcaccttcaaggagcagaacgacaagccttactgtcagaactgcttcctcaagctcttctgctag

SEQ ID NO: 13 - DNA, 20 bp, Artificial Sequence
  Primer
tgctgaagatgttggcgttg

SEQ ID NO: 14 - DNA, 21 bp, Artificial Sequence
  Primer
tgccatgctcaacatcacact

SEQ ID NO: 15 - DNA, 23 bp, Artificial Sequence
  Primer
cacttcactgtgcgcgaggcacc